(19) United States
(12) Patent Application Publication
(10) Pub. No.: US 2024/0043910 A1
(43) Pub. Date: Feb. 8, 2024

(54) METHODS AND COMPOSITIONS USING SINGLE STRAND ANNEALING PROTEINS

(71) Applicant: 10x Genomics, Inc., Pleasanton, CA (US)

(72) Inventor: Shankar SHASTRY, Pleasanton, CA (US)

(21) Appl. No.: 18/362,074

(22) Filed: Jul. 31, 2023

Related U.S. Application Data
(60) Provisional application No. 63/370,064, filed on Aug. 1, 2022.

(51) Int. Cl. C12Q 1/682; C12Q 1/6837

(52) U.S. Cl. CPC C12Q 1/682; C12Q 1/6837; C12Q 2600/16

(57) ABSTRACT

The present disclosure relates in some aspects to methods and compositions for analyzing a target nucleic acid in a biological sample. In some aspects, the methods disclosed herein promote hybridization of polynucleotides to target nucleic acids and/or splints for enhanced ligation efficiency and/or enhanced hybridization efficiency in situ in a biological sample. In some aspects, the presence, amount, and/or identity of a target nucleic acid is analyzed in situ. Also provided are compositions and kits for use in accordance with the methods.

### CROSS-REFERENCE TO RELATED APPLICATIONS

This application claims priority to U.S. Provisional Patent Application No. 63/370,064, filed Aug. 1, 2022, entitled “METHODS AND COMPOSITIONS USING SINGLE STRAND ANNEALING PROTEINS,” which is herein incorporated by reference in its entirety for all purposes.

### INCORPORATION BY REFERENCE OF SEQUENCE LISTING

The present application is being filed along with a Sequence Listing in electronic format. The Sequence Listing is provided as a file entitled 202412014800SeqList.xml, created Jul. 21, 2023, which is 8,317 bytes in size. The information in the electronic format of the Sequence Listing is incorporated by reference in its entirety.

### FIELD

The present disclosure relates in some aspects to methods and compositions for processing nucleic acid molecules, including improving nucleic acid ligation and fidelity by use of strand annealing proteins.

### BACKGROUND

Ligation to assemble nucleic acid sequences and/or for detection and analysis of target nucleic acid sequences has applications in many different fields, including cancer diagnosis, personalized medicine, and infectious diseases. However, current approaches suffer from low ligation efficiency and associated issues, such as low sensitivity for methods of analyzing of biological samples comprising ligation. Improved methods for nucleic acid ligation and/or hybridization are needed. Provided herein are methods and compositions that address these and other needs.

### SUMMARY

In some aspects, provided herein is a method for nucleic acid ligation, comprising: a) providing a first nucleic acid sequence and a second nucleic acid sequence, wherein the first nucleic acid sequence hybridizes to a first hybridization region of a third nucleic acid sequence and the second nucleic acid sequence hybridizes to a second hybridization region of the third nucleic acid sequence, wherein the first nucleic acid sequence, the second nucleic acid sequence and/or the third nucleic acid sequence are/is bound to a single strand annealing protein (SSAP); and b) ligating the first nucleic acid sequence and second nucleic acid sequence using the third nucleic acid sequence as a template to generate a ligated oligonucleotide comprising the first nucleic acid sequence and the second nucleic acid sequence.

In some embodiments, the first nucleic acid sequence and the second nucleic acid sequence are in the same nucleic acid molecule. In any of the preceding embodiments, the first nucleic acid sequence and the second nucleic acid sequence can be a first end and a second end, respectively, of a circularizable probe. In some embodiments, the first nucleic acid sequence is in a first nucleic acid strand and the second nucleic acid sequence is in a second nucleic acid strand. In some embodiments, the first nucleic acid sequence and the second nucleic acid sequence are in different nucleic acid molecules.

In any of the preceding embodiments, the single strand annealing protein may promote ligation of the first nucleic acid sequence and the second nucleic acid sequence. In any of the preceding embodiments, the single strand annealing protein may promote hybridization of the first nucleic acid sequence and/or the second nucleic acid sequence to the third nucleic acid sequence. In any of the preceding embodiments, the ligation efficiency may be increased by at least 10%, at least 20%, at least 30%, at least 40%, at least 50%, at least 60%, at least 70%, at least 80%, at least 90%, or at least 100% with the bound single strand annealing protein (e.g., the single strand annealing protein bound to the first nucleic acid sequence, the second nucleic acid sequence, and/or the third nucleic acid sequence) compared to ligation in the absence of the single strand annealing protein.

In any of the preceding embodiments, each of the first nucleic acid sequence, the second nucleic acid sequence, and the third nucleic acid sequence can be independently bound to the same single strand annealing protein or to a different single strand annealing protein. In any of the preceding embodiments, the single strand annealing protein may be DdrB or RecT. In any of the preceding embodiments, the concentration of DdrB protein used may be at least 10 nM, at least 100 nM, at least 300 nM, at least 500 nM, at least 700 nM, or at least 1000 nM.

In any of the preceding embodiments, the ligating may be performed using a ligase. In some instances, the ligase is a DNA ligase. In some instances, the ligase is an RNA ligase.

In any of the preceding embodiments, the 3′ end of the first nucleic acid sequence may be ligated to the 5′ end of the second nucleic acid sequence. In any of the preceding embodiments, the 5′ end of the first nucleic acid sequence may be ligated to the 3′ end of the second nucleic acid sequence. In any of the preceding embodiments, the first nucleic acid sequence can be in a probe, and the method can comprise contacting a biological sample with the probe, wherein the third nucleic acid sequence is in a target nucleic acid in the biological sample, wherein the second nucleic acid sequence is in the same probe or is in a different probe that hybridizes to the target nucleic acid, wherein the ligating is performed in the biological sample, and wherein the ligated oligonucleotide is a ligated probe comprising the first nucleic acid sequence and the second nucleic acid sequence.

In any of the preceding embodiments, the method can comprise detecting the ligated probe or a product thereof. In any of the preceding embodiments, the method can comprise detecting the ligated probe or a product thereof at a location in the biological sample.

In any of the preceding embodiments, the second hybridization region can be adjacent to the first hybridization region in the third nucleic acid sequence. In any of the preceding embodiments, the first hybridization region and the second hybridization region in the third nucleic acid sequence can be directly linked by a phosphodiester bond. In any of the preceding embodiments, the second hybridization region may not be directly adjacent to the first hybridization region in the third nucleic acid sequence. In some embodiments, the first hybridization region and the second hybridization region in the third nucleic acid sequence are linked by one, two, three, four, five, six, seven, eight, nine, 10, or more nucleic acid residues. In some embodiments, the method comprises performing a gap filling reaction with a polymerase using the third nucleic acid sequence as a template and/or hybridizing an oligonucleotide to the third nucleic acid sequence at a region between the first and second hybridization regions. In any of the preceding embodiments, the ligated oligonucleotide can be a linear probe or a circular probe.

In any of the preceding embodiments, the third nucleic acid sequence can be RNA, and the first nucleic acid sequence and/or second nucleic acid sequence can comprise DNA. In any of the preceding embodiments, the first nucleic acid sequence and/or the second nucleic acid sequence can comprise a ribonucleotide, optionally wherein the ribonucleotide is a 3′ end ribonucleotide of the first nucleic acid sequence or the second nucleic acid sequence that is ligated to the second nucleic acid sequence or the first nucleic acid sequence, respectively. In any of the preceding embodiments, the first nucleic acid sequence and/or the second nucleic acid sequence may comprise no more than four consecutive ribonucleotides. In some embodiments, the 5′ nucleotide of the first nucleic acid sequence and/or the 5′ nucleotide of the second nucleic acid sequence is a deoxyribonucleoside comprising a 5′ phosphate.

In any of the preceding embodiments, the first nucleic acid sequence may be in a first nucleic acid strand comprising a first barcode sequence. In any of the preceding embodiments, wherein the second nucleic acid sequence may be in a second nucleic acid strand comprising a second barcode sequence. The first and second barcode sequences can be of the same sequence or of different sequences. In some embodiments, the first barcode sequence is in a 3′ overhang region of the first nucleic acid strand upon hybridization to the third nucleic acid sequence, and the second barcode sequence is in a 5′ overhang region of the second nucleic acid strand upon hybridization to the third nucleic acid sequence. In some embodiments, the first barcode sequence is in a 5′ overhang region of the first nucleic acid strand upon hybridization to the third nucleic acid sequence, and the second barcode sequence is in a 3′ overhang region of the second nucleic acid strand upon hybridization to the third nucleic acid sequence. In any of the preceding embodiments, the nucleic acid molecule, probe, or first and second probe can comprise a barcode sequence. In any of the preceding embodiments, the first nucleic acid sequence and/or the second nucleic acid sequence can comprise a barcode sequence or a portion thereof.

In any of the preceding embodiments, the biological sample can be further contacted with a secondary probe that hybridizes to the ligated probe or a product thereof. In any of the preceding embodiments, the secondary probe can be a detectably labeled probe comprising a detectable moiety. In any of the preceding embodiments, the secondary probe can comprise an overhang region that does not hybridize to the ligated probe or product thereof. In any of the preceding embodiments, the method can comprise contacting the biological sample with a detectably labeled oligonucleotide, wherein the detectably labeled oligonucleotide comprises a detectable moiety and a sequence that hybridizes to the overhang region of the secondary probe. In any of the preceding embodiments, the detectable moiety can be a fluorophore.

In any of the preceding embodiments, the secondary probe can be bound to a single strand annealing protein. In some embodiments, the single strand annealing protein promotes hybridization of the secondary probe to the ligated probe or product thereof. In any of the preceding embodiments, the detectably labeled oligonucleotide can be bound to a single strand annealing protein. In some embodiments, the single strand annealing protein promotes hybridization of the detectably labeled oligonucleotide to the secondary probe.

In any of the preceding embodiments, the method can comprise detecting the ligated probe or product thereof. In some embodiments, the detecting comprises determining a sequence of the probe, or a complementary sequence or a product of the probe. In any of the preceding embodiments, the product can be a hybridization product, a ligation product, an extension product (e.g., by a DNA or RNA polymerase), a replication product, a transcription/reverse transcription product, and/or an amplification product. In any of the preceding embodiments, the target nucleic acid can at a location in a biological sample and the ligated probe can generated and optionally amplified at the location in the biological sample. In any of the preceding embodiments, the ligated probe and/or the product thereof may be detected at the location in the biological sample. In some instances, the product of the ligated probe is a rolling circle amplification (RCA) product (e.g., wherein the ligated probe is a circularized probe).

In some embodiments, the method comprises capturing the ligated probe on an array. In some embodiments, the method comprises sequencing all or a portion of the captured ligated probe or a complement thereof. In some embodiments, the captured ligated probe or complement thereof are amplified. In any of the preceding embodiments, the amplification product of the captured ligated probe or complement thereof comprises a spatial barcode sequence or a complement thereof that identifies the location of the captured ligated probe on the array.

In any of the preceding embodiments, the first nucleic acid sequence, the second nucleic acid sequence, and the third nucleic acid sequence may be DNA. In any of the preceding embodiments, the second nucleic acid sequence and the third nucleic acid sequence may be provided in a) as a partially double stranded duplex.

In any of the preceding embodiments, the first nucleic acid strand can immobilized on a substrate. In any of the preceding embodiments, the first nucleic acid strand may comprise a primer sequence. In any of the preceding embodiments, the first nucleic acid strand may further comprise a unique molecular identifier (UMI). In any of the preceding embodiments, the first nucleic acid strand can further comprise a barcode sequence or portion thereof. In any of the preceding embodiments, the second nucleic acid sequence can comprise a barcode sequence or portion thereof. In some instances, the barcode sequence is a spatial barcode associated with a location on the substrate. In any of the preceding embodiments, the second nucleic acid strand can further comprise a splint sequence. In some embodiments, the splint sequence can be common among a plurality of second nucleic acid strands immobilized on the substrate or on a plurality of substrates.

In any of the preceding embodiments, the method can further comprise providing a fourth nucleic acid strand and a fifth nucleic acid strand, wherein the splint sequence of the second nucleic acid strand hybridizes to a third hybridization region in the fifth nucleic acid strand, and the fourth nucleic acid strand comprises a fourth nucleic acid sequence that hybridizes to a fourth hybridization region in the fifth nucleic acid strand. In some embodiments, the second, fourth, and/or fifth nucleic acid strand are/is bound to a single strand annealing protein. In any of the preceding embodiments, the fourth nucleic acid strand can comprise a barcode sequence or portion thereof. In some embodiments, the barcode sequence of the fourth nucleic acid strand is a spatial barcode associated with a location on the substrate. In any of the preceding embodiments, the fourth nucleic acid strand can comprise a second primer sequence. In any of the preceding embodiments, the fourth nucleic acid strand can comprise a second unique molecular identifier sequence. In any of the preceding embodiments, the second nucleic acid strand may be ligated to the fourth nucleic acid strand.

In any of the preceding embodiments, the single strand annealing protein may promote ligation of the second nucleic acid strand to the fourth nucleic acid strand. In any of the preceding embodiments, the single strand annealing protein may promote hybridization of the second and/or fourth nucleic acid strand to the fifth nucleic acid strand.

In any of the preceding embodiments, the method can comprise generating a ligated oligonucleotide comprising the first nucleic acid sequence, the second nucleic acid sequence, and the fourth nucleic acid sequence. In any of the preceding embodiments, the method can comprise providing a sixth nucleic acid strand and a seventh nucleic acid strand, wherein the fourth nucleic acid strand comprises a splint sequence that hybridizes to a sixth hybridization region in the seventh nucleic acid strand, and the sixth nucleic acid strand comprises a sixth nucleic acid sequence that hybridizes to a seventh hybridization region in the seventh nucleic acid strand. In some instances, the fourth, sixth, and/or seventh nucleic acid strand are/is bound to a single strand annealing protein. In any of the preceding embodiments, the fourth nucleic acid strand may be ligated to the sixth nucleic acid strand.

In any of the preceding embodiments, the method can comprise N cycles of ligation of different nucleic acid strands to the first nucleic acid strand to generate an immobilized ligated nucleic acid, wherein N is an integer of 2 or greater, and one of the N cycles comprises the providing in step a) and ligating in step b). In any of the preceding embodiments, at least 2, 3, or 4 of the N cycles may be performed in the presence of the single strand annealing protein. In any of the preceding embodiments, a plurality of first nucleic acid strands may be immobilized in a plurality of features on the substrate. In any of the preceding embodiments, the nucleic acid sequences received by the first nucleic acid strands on the substrate in cycle I and in cycle J can be different, wherein I and J are integers and 1≤I<J≤N. In any of the preceding embodiments, the immobilized ligated nucleic acid may comprise a capture sequence.

In any of the preceding embodiments, the substrate can be a planar substrate. In any of the preceding embodiments, the substrate can be a bead. In any of the preceding embodiments, the bead can be a gel bead.

In any of the preceding embodiments, the method can comprise ligating a second nucleic acid strand A to the immobilized first nucleic acid strand for a plurality of beads in a partition A, and ligating a different second nucleic acid strand B to the immobilized first nucleic acid strand for a plurality of beads in a partition B; pooling partitions A and B; and splitting the pooled partitions into partitions C and D before performing one or more additional cycles of ligating nucleic acid strands to the immobilized nucleic acid strands in the partitions, wherein each of the cycles of ligating is performed in the presence of a single strand annealing protein.

In some aspects, provided herein is a method for analyzing a target RNA, comprising: a) contacting the target RNA with a circularizable probe or probe set comprising a first nucleic acid sequence that hybridizes to a first hybridization region of the target RNA; b) ligating the first nucleic acid sequence of the circularizable probe or probe set to a second nucleic acid sequence hybridized to a second hybridization region of the target RNA in the presence of a single strand annealing protein bound to the first nucleic acid sequence and/or the second nucleic acid sequence, wherein the second nucleic acid sequence is a part of the circularizable probe or probe set, wherein the second hybridization region is adjacent to the first hybridization region in the target RNA, thereby generating a circularized probe comprising the first nucleic acid sequence and the second nucleic acid sequence; and c) detecting the circularized probe or a product thereof. In any of the preceding embodiments, the circularizable probe or probe set can be a DNA probe or probe set or can comprise primarily of DNA residues. In any of the preceding embodiments, the circularizable probe or probe set can comprise one or more RNA residues. In some embodiments, the one or more RNA residues can comprise a 3′ end ribonucleotide of the circularizable probe or probe set. In any of the preceding embodiments, the circularizable probe or probe set may comprise no more than four consecutive RNA residues. In any of the preceding embodiments, a 5′ nucleotide of the circularizable probe or probe set is a DNA residue comprising a 5′ phosphate that can be ligated to the 3′ end ribonucleotide. In any of the preceding embodiments, the product of the circularized probe can be a rolling circle amplification (RCA) product. In any of the preceding embodiments, the circularized probe or product (e.g., RCA product) thereof can be detected at a location in a biological sample (e.g., a cell or tissue sample), thereby detecting the target RNA at the location in the biological sample.

In some aspects, provided herein is a method for analyzing a biological sample, comprising: contacting a target nucleic acid in the biological sample with a probe, wherein the probe is bound to a single strand annealing protein and the probe comprises a recognition sequence that hybridizes to a target sequence in the target nucleic acid, and detecting a signal associated with the probe, thereby detecting the target nucleic acid in the biological sample. In some aspects, provided herein is a method for analyzing a biological sample, comprising: contacting a target nucleic acid in the biological sample with a probe, wherein the probe is bound to a single strand annealing protein and the probe comprises a recognition sequence that hybridizes to a target sequence in the target nucleic acid, and detecting a signal associated with the probe at a location in the biological sample, thereby detecting the target nucleic acid at the location in the biological sample. In any of the preceding embodiments, the probe can be a detectably labeled probe. In any of the preceding embodiments, the probe can comprise a binding site for a detectably labeled oligonucleotide, or for an intermediate probe that binds directly or indirectly to a detectably labeled oligonucleotide, and the method can comprise contacting the biological sample with the detectably labeled oligonucleotide and/or intermediate probe, thereby associating the signal with the probe hybridized to the target nucleic acid. In any of the preceding embodiments, the target nucleic acid may be a DNA concatemer comprising multiple copies of the target sequence. In any of the preceding embodiments, the target nucleic acid can be an RNA.

In some aspects, provided herein is a method comprising generating a plurality of ligated oligonucleotides, wherein each ligated oligonucleotide is generated according to the method of any of the preceding embodiments, and detecting the plurality of ligated oligonucleotides or products thereof. In some embodiments, the plurality of ligated oligonucleotides or products thereof are detected at different locations in the biological sample. In some embodiments, the detecting comprises: contacting the biological sample with one or more detectably labeled probes that directly or indirectly bind to one or more barcode sequences or complements thereof in the plurality of ligated oligonucleotides or products thereof, detecting signals associated with the one or more detectably labeled probes, and removing the one or more detectably labeled probes. In some embodiments, the detecting comprises: contacting the biological sample with one or more intermediate probes that directly or indirectly bind to one or more barcode sequences or complements thereof in the plurality of ligated oligonucleotides or products thereof, wherein the one or more intermediate probes are detectable using one or more detectably labeled probes, and detecting signals associated with the one or more detectably labeled probes. In any of the preceding embodiments, the method can comprise removing the one or more intermediate probes and/or the one or more detectably labeled probes.

In some embodiments, the method can comprise capturing the plurality of ligated oligonucleotides on an array. In some embodiments, detecting the plurality of ligated oligonucleotides or products thereof comprises sequencing all or a portion of the captured ligated oligonucleotides or complements thereof. In some embodiments, the captured ligated oligonucleotides or complements thereof are amplified. In any of the preceding embodiments, the amplification products of the captured ligated oligonucleotides or complements thereof can comprise spatial barcode sequences or complements thereof that identify locations of the captured ligated oligonucleotides on the array.

### BRIEF DESCRIPTION OF THE DRAWINGS

The drawings illustrate certain features and advantages of this disclosure. These embodiments are not intended to limit the scope of the appended claims in any manner.

FIG. 1 depicts an example of a method comprising ligation of a first and a second nucleic acid strand using a third nucleic acid strand as a template, wherein single strand annealing protein molecules are bound to the first, second, and/or third nucleic acid strand.

FIG. 2A depicts an example of a method for ligating a circularizable probe comprising a first nucleic acid sequence and a second nucleic acid sequence using a third nucleic acid sequence as a template, wherein the circularizable probe is bound to single strand annealing protein molecules. FIG. 2B depicts an example of a method for ligating a probe set comprising a first nucleic acid sequence and a second nucleic acid sequence using a third nucleic acid sequence as a template, wherein the first probe and the second probe of the probe set are both bound to single strand annealing protein molecules.

FIG. 3 depicts an example of a method of analyzing a biological sample comprising contacting the biological sample with detectably labeled probes bound to single strand annealing proteins, wherein the single strand annealing proteins promote the annealing of the detectably labeled probes to complementary sequences on a rolling circle amplification product.

FIG. 4 depicts an example of a method of generating a barcoded oligonucleotide immobilized on a substrate (e.g., a planar substrate or a bead) using multiple cycles of ligation assisted by a single strand annealing protein.

FIGS. 5A-5B show a schematic of a ligation assay (FIG. 5A) and the results of a capillary electrophoresis reaction (FIG. 5B) to determine the effect of single strand annealing protein DdrB on the ligation efficiency of a first and second nucleic acid strand of DNA using a third nucleic acid strand of DNA as a template (DNA-templated ligation e.g., DNA probe hybridized to a DNA target).

FIG. 6 provides results demonstrating improved ligation efficiency (%) of DNA templated DNA ligation reactions in the presence of single strand annealing protein DdrB at varying concentrations.

FIGS. 7A-7B show a schematic of a ligation assay (FIG. 7A) and the results of a capillary electrophoresis reaction (FIG. 7B) to determine the effect of single strand annealing protein DdrB on the ligation efficiency of a first and second nucleic acid strand of DNA using a third nucleic acid strand of RNA as a template (RNA-templated ligation, e.g., DNA probes hybridized to an RNA target).

FIG. 8 shows improved ligation efficiency (%) of RNA-templated DNA ligation reactions in the presence of single strand annealing protein DdrB at varying concentrations compared to ligation in the absence of DdrB.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Nucleic acid hybridization and ligation is used for a wide variety of applications. For example, accurate hybridization of nucleic acid probes is used to analyze nucleic acid sequences, including analyzing target nucleic acids in situ in a biological sample (e.g., at spatially localized positions in a biological sample). In some cases, methods of analyzing target nucleic acid molecules in a biological sample comprise ligating one or more probes. In some instances, two nucleic acid probes are ligated together in a templated ligation that requires accurate hybridization of both nucleic acid probes to adjacent regions of a target nucleic acid. In other cases, the 3′ and 5′ ends of a circularizable probe such as a padlock probe are ligated together using a target nucleic acid or other probe (e.g., a target RNA or a target DNA) as a template to generate a circular probe, which can be used as a template for rolling circle amplification. In some cases, such methods suffer from low hybridization fidelity and/or efficiency. Furthermore, methods that depend on ligation (e.g., to generate a ligated product or an amplification product thereof that is detected in a biological sample) may suffer from low ligation efficiency, resulting in low sensitivity.

Ligation efficiency is also important for methods that rely on ligation to process nucleic acids during manufacturing of ligated oligonucleotides, such as in combinatorial barcoding using ligation. In some aspects, provided herein are methods to improve ligation efficiency and/or fidelity using single strand DNA annealing proteins for the manufacture of barcoded molecules. In some cases, the methods comprise multiple rounds or cycles of ligation (e.g., attaching multiple nucleic acid strands to a first nucleic acid strand in sequential ligations). Notably, the effect of increased ligation efficiency can be multiplied across multiple rounds of ligation, resulting in a dramatic increase in the efficiency of generating full-length barcoded oligonucleotides in methods comprising multiple rounds of ligation. For example, in some cases increasing the ligation efficiency for each round of ligation from 40% to 80% in a method comprising three rounds of ligation results in an improvement in the efficiency of generating full-length ligated oligonucleotides (comprising the nucleic acid strands ligated in all three rounds of ligation) from about 6% (0.4×0.4×0.4) to about 50% (0.8×0.8×0.8), increasing the overall efficiency of generating full-length ligated oligonucleotides by more than 5-fold.

The present application provides methods of improving the efficiency and/or fidelity of nucleic acid ligation using single strand annealing proteins (SSAP). Single stranded DNA annealing proteins are critical components of certain DNA homologous recombination systems. For example, the Escherichia coli single strand annealing protein RecT and the Deinococcus single strand annealing protein DdrB (named for DNA damage response B) promote single strand annealing and repair of double strand breaks in cells. The present disclosure in some aspects provides methods for ligating nucleic acid strands wherein one or more of the nucleic acid strands are bound by single strand annealing proteins such as RecT or DdrB. In other aspects, the present disclosure provides methods for improved probe hybridization in situ in a biological sample, wherein the probe(s) are bound by single strand annealing protein(s). Furthermore, the present application provides data demonstrating significantly increased ligation efficiency for DNA strands using a DNA template in the presence of a single strand annealing protein, and results demonstrating significantly increased ligation efficiency for DNA strands using an RNA template in the presence of a single strand annealing protein. In some aspects, ligation efficiencies during DNA templated or RNA templated ligation reactions may be significantly improved by using strand annealing proteins such as DdrB or RecT. In some embodiments, single strand annealing protein-assisted DNA strands are used for annealing to RNA in applications involving RNA-templated ligation (e.g., DNA probes hybridized to RNA targets, such as circularizable probes hybridized to target mRNA). Furthermore, strand annealing protein-assisted oligonucleotide annealing to DNA may be used for applications involving annealing of short complementary oligonucleotides during generation of spatial arrays. Strand annealing protein-assisted annealing may also be used for applications involving detection of hybridization products (e.g., rolling circle amplification products). For example, single stranded DNA annealing protein-assisted oligonucleotide annealing may be used to decode rolling circle amplification products in situ, as an approach to in situ detection. In some embodiments, the single strand annealing protein is DdrBor RecT. In some embodiments, the ligation efficiency is increased by at least 10%, at least 20%, at least 30%, at least 40%, at least 50%, at least 60%, at least 70%, at least 80%, at least 90%, or by at least 100%. In some embodiments, the hybridization efficiency is increased by at least 10%, at least 20%, at least 30%, at least 40%, at least 50%, at least 60%, at least 70%, at least 80%, at least 90%, or at least 100%. In some embodiments, the ligation efficiency is increased by at least 2-fold, at least 2.5-fold, or at least 3-fold. In some embodiments, the hybridization efficiency is increased by at least 2-fold, at least 2.5-fold, or at least 3-fold. In some embodiments, the single strand annealing protein (e.g., DdrB or RecT) binds to single stranded nucleic acid strands (e.g., probes, splints, or nucleic acid barcode components) and facilitate their annealing to target nucleic acid molecules such as rolling circle amplification products (RCPs), RNA templates, or other oligonucleotides (e.g., for spatial array synthesis).

In some aspects, provided herein is a method for nucleic acid ligation, comprising a) providing a first nucleic acid sequence and a second nucleic acid sequence, wherein the first nucleic acid sequence hybridizes to a first hybridization region of a third nucleic acid sequence and the second nucleic acid sequence hybridizes to a second hybridization region of the third nucleic acid sequence, wherein the first nucleic acid sequence, the second nucleic acid sequence and/or the third nucleic acid sequence are/is bound to a single strand annealing protein; and b) ligating the first nucleic acid sequence and second nucleic acid sequence using the third nucleic acid sequence as a template to generate a ligated oligonucleotide comprising the first nucleic acid sequence and the second nucleic acid sequence. In some embodiments, the ligation is performed in situ in a biological sample. In some embodiments, the first nucleic acid sequence and second nucleic acid sequence are in a first and second nucleic acid strand, respectively. In some embodiments, the first and second nucleic acid strands are first and second probes, and the third nucleic acid sequence is in a target nucleic acid in the biological sample (e.g., an endogenous nucleic acid or product thereof, or a nucleic acid sequence in a probe or labelling agent that binds directly or indirectly to a nucleic acid or non-nucleic acid analyte in the biological sample). In some embodiments, the first nucleic acid sequence and second nucleic acid sequence are in the same nucleic acid molecule (e.g., at first and second end of a circularizable probe). The ends of the circularizable probe hybridize to the third nucleic acid sequence such that the 3′ end and 5′ end are ligatable using the third nucleic acid sequence as a template, with or without gap filling prior to ligation. In some embodiments, the method results in increased ligation efficiency compared to a ligation performed under the same conditions but in the absence of a single strand annealing protein. In some embodiments, the method results in increased ligation fidelity compared to a ligation performed under the same conditions but in the absence of a single strand annealing protein.

In some aspects, the methods provided herein comprise ligating a first nucleic acid strand immobilized on a substrate to a second nucleic acid strand using a third nucleic acid strand (splint) as a template to generate a ligated oligonucleotide immobilized on the substrate, wherein the first nucleic acid strand, second nucleic acid strand, and/or third nucleic acid strand are bound to single strand annealing proteins. In some embodiments, the method comprises performing multiple sequential rounds of ligation to ligate the same or different nucleic acid sequences to the immobilized ligated oligonucleotide in the presence of a single strand annealing protein. Also described herein are methods comprising performing multiple ligations in a combinatorial scheme to combinatorially generate a high diversity of different ligated oligonucleotides on different regions of a substrate or on different substrates (e.g., beads). In some embodiments, the method results in increased ligation efficiency compared to a ligation performed under the same conditions but in the absence of a single strand annealing protein. In some embodiments, the method results in increased ligation fidelity compared to a ligation performed under the same conditions but in the absence of a single strand annealing protein.

In some aspects, provided herein is a method for analyzing a biological sample, comprising contacting a target nucleic acid in the biological sample with a probe, wherein the probe is bound to a single strand annealing protein and the probe comprises a recognition sequence that hybridizes to a target sequence in the target nucleic acid, detecting a signal associated with the probe, thereby detecting the target nucleic acid in the biological sample. In some embodiments, the probe is a detectably labeled probe (e.g., the probe may comprise a detectable moiety such as a fluorophore). In some embodiments, the probe comprises a binding site for a detectably labeled oligonucleotide associated with the signal, or for an intermediate probe that binds directly or indirectly to a detectably labeled oligonucleotide, and the method comprises contacting the biological sample with the detectably labeled oligonucleotide and/or intermediate probe, thereby associating the signal with the probe hybridized to the target nucleic acid. In some embodiments, the target nucleic acid is a DNA concatemer comprising multiple copies of the target sequence. In some embodiments, the target nucleic acid is an RNA. In some instances, the method comprises contacting the target nucleic acid (e.g., an mRNA) with a plurality of probes comprising recognition sequences complementary to a plurality of target sequences in the target nucleic acid, wherein the probes are bound to single strand annealing proteins. In some embodiments, the probes comprise overhang regions that do not hybridize to the target nucleic acid, and the method comprises contacting the biological sample with a pool of detectably labeled oligonucleotides that hybridize to corresponding overhang regions in the probes, thereby associating the signal with the probes. In some cases, the detectably labeled oligonucleotides are bound to single strand annealing proteins. In some embodiments, the method results in increased hybridization efficiency compared to hybridization of the same probes or detectably labeled oligonucleotides under the same conditions but in the absence of the single strand annealing protein. In some embodiments, the method reduces off-target hybridization of the probes or detectably labeled oligonucleotides under the same conditions but in the absence of the single strand annealing protein.

In certain aspects, the methods, compositions, and kits disclosed herein can be used to improve the ligation efficiencies of polynucleotides in a multitude of in situ detection applications, including but not limited to ligation of single stranded DNA splinted by complementary RNA sequences, detection of RNA using ligation of DNA probes, detection of single nucleotide polymorphisms (SNPs) or splice variants in RNA or cDNA using ligation of nucleic acid probes, RNA-mediated oligonucleotide annealing, selection, and ligation with next-generation sequencing (RASL-Seq), and circularizable probe or probe set (e.g., padlock probe) based RNA detection. The single strand annealing protein-bound polynucleotides can be used to improve ligation efficiencies in spatial array-based assays to identify the spatial locations of targets in a biological sample, or for single cell profiling assays coupled with sequencing (e.g., next-generation sequencing).

### II. Methods

In some aspects, provided herein is a method for nucleic acid annealing and/or ligation, comprising providing a first nucleic acid sequence and a second nucleic acid sequence, wherein the first nucleic acid sequence hybridizes to a first hybridization region of a third nucleic acid sequence and the second nucleic acid sequence hybridizes to a second hybridization region of the third nucleic acid sequence, wherein the first nucleic acid sequence, second nucleic acid sequence and/or the third nucleic acid are/is bound to a single strand annealing protein; and ligating the first nucleic acid sequence and second nucleic acid sequence using the third nucleic acid sequence as a template to generate a ligated oligonucleotide comprising the first nucleic acid sequence and the second nucleic acid sequence. In some embodiments, the first nucleic acid sequence and the second nucleic acid sequence are in the same nucleic acid molecule. In some embodiments, the first nucleic acid sequence is in a first nucleic acid strand and the second nucleic acid sequence is in a second nucleic acid strand. In some embodiments, the first nucleic acid strand is immobilized on a substrate. In some embodiments, the first nucleic acid sequence and the second nucleic acid sequence are in a single probe (e.g., a circularizable probe) or a first and second probe, respectively. In some aspects, provided herein is a method for analyzing a biological sample comprising hybridizing one or more probes to one or more target nucleic acids in the biological sample in the presence of a single strand annealing protein. In some embodiments, the method comprises contacting the sample with the single strand annealing protein.

The nucleic acid sequences may comprise any one of a variety of entities that can hybridize to a nucleic acid, typically by Watson-Crick base pairing, such as DNA, RNA, LNA, PNA, etc. The nucleic acid sequence typically contains a region that is able to bind and/or hybridize to at least a portion of a complementary nucleic acid sequence (e.g., in another nucleic acid strand). In some aspects, the method comprises providing a first nucleic acid sequence and a second nucleic acid sequence, wherein the first nucleic acid sequence hybridizes to a first hybridization region of a third nucleic acid sequence and the second nucleic acid sequence hybridizes to a second hybridization region of the third nucleic acid sequence. In some embodiments, the first nucleic acid sequence is bound by a single strand annealing protein. In some embodiments, the second nucleic acid sequence is bound by a single strand annealing protein. In some embodiments, the single strand annealing protein is RecT, Redβ, DdrB, or a variant or derivative of any of the aforementioned proteins.

In some embodiments of the methods disclosed herein, the first nucleic acid strand or sequence is bound to a single strand annealing protein. In some embodiments of the methods disclosed herein, the second nucleic acid strand or sequence is bound to a single strand annealing protein. In some embodiments of the methods disclosed herein, the third nucleic acid strand or sequence is bound to a single strand annealing protein. In some embodiments, a single stranded portion of the first, second, and/or third nucleic acid sequence is bound to a single strand annealing protein. In some aspects, a circularizable probe disclosed herein is bound to a single strand annealing protein. In some aspects, the first and second nucleic acid sequences of the disclosed probes are bound to single strand annealing proteins. In some aspects, the first nucleic acid sequence and the third nucleic acid sequence are bound to single strand annealing proteins. In some embodiments, the second nucleic acid sequence and the third nucleic acid sequence are bound to single strand annealing proteins. In some embodiments, the first nucleic acid sequence, the second nucleic acid sequence, and the third nucleic acid sequence are bound to single strand annealing proteins.

In some embodiments, the concentration of single strand annealing protein used is at least 100 nM, at least 300 nM, at least 500 nM, at least 700 nM, at least 1000 nM. In some embodiments, the concentration of SSAP used is at least 100 nM, at least 150 nM, at least 200 nM, at least 250 nM, at least 300 nM, at least 350 nM, at least 400 nM, at least 450 nM, at least 500 nM, at least 550 nM, at least 600 nM, at least 650 nM, at least 700 nM, at least 750 nM, at least 800 nM, at least 850 nM, at least 900 nM, at least 950 nM, or at least 1000 nM.

In some embodiments, the single strand annealing protein is a RecT protein or an engineered variant or derivative thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to the sequence of SEQ ID NO: 1. In some embodiments, the single strand annealing protein comprises the amino acid sequence of SEQ ID NO: 1. In some embodiments, the single strand annealing protein further comprises one or more tags (e.g., a purification tag such as a polyhistidine tag). In some embodiments, the single strand annealing protein comprises a deletion of the amino terminal methionine (e.g., a truncation of M1, wherein the amino acid numbering is based on SEQ ID NO: 1).

In some embodiments, the single strand annealing protein is a Lambda phage Redβ protein or an engineered variant or derivative thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to the sequence of SEQ ID NO: 2. In some embodiments, the single strand annealing protein comprises the amino acid sequence of SEQ ID NO: 2. In some embodiments, the single strand annealing protein further comprises one or more tags (e.g., a purification tag such as a polyhistidine tag). In some embodiments, the single strand annealing protein comprises a deletion of the amino terminal methionine (e.g., a truncation of M1, wherein the amino acid numbering is based on SEQ ID NO: 2).

In some embodiments, the single strand annealing protein is DdrB or an engineered variant or derivative thereof (e.g., a polypeptide having at least 50%, at least 60%, at least 70%, at least 80%, at least 85%, at least 90%, or at least 95% amino acid sequence identity). DdrB is a single strand annealing protein derived from Deinococcus bacteria. The DdrB can be a DdrB from any species of Deinococcus, or an engineered variant or derivative thereof. In some embodiments, the DdrB is Deinococcus radiodurans (D. radiodurans) DdrB, D. geothermalis DdrB, or D. deserti DdrB, or an engineered variant thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to the sequence of any of SEQ ID NOs: 3-5. In some embodiments, the single strand annealing protein comprises the amino acid sequence of any of SEQ ID NOs: 3-5. In some embodiments, the single strand annealing protein further comprises one or more tags (e.g., a purification tag such as a polyhistidine tag). In some embodiments, the single strand annealing protein comprises a deletion of the amino terminal methionine (e.g., a truncation of M1, wherein the amino acid numbering is based any one of SEQ ID NOs: 3-5).

In some embodiments, the single strand annealing protein is D. radiodurans DdrB or an engineered variant or derivative thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to SEQ ID NO. 3. In some embodiments, the single strand annealing protein comprises amino acid residues that are conserved among SEQ ID NOs: 3-5. In some embodiments, the strand annealing protein comprises one or more mutations (e.g., substitutions, deletions, or insertions) in sequences that are not-conserved among SEQ ID NOs: 3-5. Conserved residues can be identified by multiple sequence alignment. In some embodiments, the single strand annealing protein comprises a sequence having at least about 80%, at least about 90%, at least about 95%, at least about 96%, at least about 97%, at least about 98%, or at least about 99% amino acid sequence identity to the sequence of SEQ ID NO: 3, wherein amino acid residues 1, 3-5, 7-11, 13-15, 17-18, 23-28, 30-38, 41, 43-48, 51-52, 54-65, 68, 70-72, 76, 78-93, 95-99, 101, 103-107, 110-111, 113, 115-120, 123-125, 127-143, 173-175, and 183-187 of SEQ ID NO: 3 are conserved. In some embodiments, the single strand annealing protein comprises the amino acid sequence of SEQ ID NO: 3. In some embodiments, the single strand annealing protein further comprises one or more tags (e.g., a purification tag such as a polyhistidine tag). In some embodiments, the single strand annealing protein comprises a deletion of the amino terminal methionine (e.g., a truncation of M1, wherein the amino acid numbering is based on SEQ ID NO: 3).

In some embodiments, the single strand annealing protein is D. geothermalis DdrB or an engineered variant or derivative thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to SEQ ID NO. 4. In some embodiments, the single strand annealing protein comprises amino acid residues that are conserved among SEQ ID NOs: 3-5. In some embodiments, the strand annealing protein comprises one or more mutations (e.g., substitutions, deletions, or insertions) in sequences that are not-conserved among SEQ ID NOs: 3-5. Conserved residues can be identified by multiple sequence alignment. In some embodiments, the single strand annealing protein comprises a sequence having at least about 80%, at least about 90%, at least about 95%, at least about 96%, at least about 97%, at least about 98%, or at least about 99% amino acid sequence identity to the sequence of SEQ ID NO: 4, wherein amino acid residues 1, 3-5, 7-11, 13-15, 17-18, 23-28, 30-38, 41, 43-48, 51-52, 54-65, 68, 70-72, 76, 78-93, 95-99, 101, 103-107, 110-111, 113, 115-120, 123-125, 127-143, 163-165, and 173-177 of SEQ ID NO: 4 are conserved. In some embodiments, the single strand annealing protein comprises the amino acid sequence of SEQ ID NO: 4. In some embodiments, the single strand annealing protein further comprises one or more tags (e.g., a purification tag such as a polyhistidine tag). In some embodiments, the single strand annealing protein comprises a deletion of the amino terminal methionine (e.g., a truncation of M1, wherein the amino acid numbering is based on SEQ ID NO: 4).

In some embodiments, the single strand annealing protein is D. deserti DdrB or an engineered variant or derivative thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to SEQ ID NO. 5. In some embodiments, the single strand annealing protein comprises amino acid residues that are conserved among SEQ ID NOs: 3-5. In some embodiments, the strand annealing protein comprises one or more mutations (e.g., substitutions, deletions, or insertions) in sequences that are not-conserved among SEQ ID NOs: 3-5. Conserved residues can be identified by multiple sequence alignment. In some embodiments, the single strand annealing protein comprises a sequence having at least about 80%, at least about 90%, at least about 95%, at least about 96%, at least about 97%, at least about 98%, or at least about 99% amino acid sequence identity to the sequence of SEQ ID NO: 5, wherein amino acid residues 1, 3-5, 7-11, 13-15, 17-18, 23-28, 30-38, 41, 43-48, 51-52, 54-65, 68, 70-72, 76, 78-93, 95-99, 101, 103-107, 110-111, 113, 115-120, 123-125, 127-143, 163-165, and 173-177 of SEQ ID NO: 5 are conserved. In some embodiments, the single strand annealing protein comprises the amino acid sequence of SEQ ID NO: 5. In some embodiments, the single strand annealing protein further comprises one or more tags (e.g., a purification tag such as a polyhistidine tag). In some embodiments, the single strand annealing protein comprises a deletion of the amino terminal methionine (e.g., a truncation of M1, wherein the amino acid numbering is based on SEQ ID NO: 5).

In some embodiments, the annealing of a single-stranded nucleic acid sequence (e.g., a first and/or second nucleic acid strand or a first and/or second nucleic acid sequence of a circularizable probe such as a padlock probe, SNAIL probe, or ROLLFISH probe) to a single-stranded target nucleic acid is promoted by the strand annealing protein binding to the single-stranded nucleic acid sequence and/or target nucleic acid. The single-stranded target nucleic acid can be an RNA molecule (e.g., an endogenous mRNA in a biological sample), a cDNA molecule, or a probe or labeling agent (e.g., a primer that hybridizes to a nucleic acid analyte in a biological sample, wherein the primer comprises an overhang region). In some embodiments, the annealing (e.g., hybridization) of a single-stranded nucleic acid sequence (e.g., a first and/or second nucleic acid strand or a first and/or second nucleic acid sequence of a circularizable probe such as a padlock probe, SNAIL probe, or ROLLFISH probe) to a single-stranded target nucleic acid is promoted by strand annealing proteins binding to the single-stranded nucleic acid sequence and the target nucleic acid sequence.

In some embodiments, the reaction kinetics (e.g., speed) of the annealing (e.g., hybridization) of the polynucleotides (e.g., hybridization of the first nucleic acid sequence to the third nucleic acid sequence and/or hybridization of the second nucleic acid sequence to the third nucleic acid sequence) are dependent on the strand annealing protein. In some embodiments, the annealing of the polynucleotides (e.g., hybridization of the first nucleic acid sequence to the third nucleic acid sequence and/or hybridization of the second nucleic acid sequence to the third nucleic acid sequence) is faster when the sample is contacted with the strand annealing protein as compared to when it is not contacted with the strand annealing protein. In some embodiments, the annealing of polynucleotides and target nucleic acid is faster when the sample is contacted with the strand annealing protein and the nucleic acid probe and/or the target nucleic acid are bound to a single stranded binding proteins, as compared to when the sample is not contacted with the strand annealing protein and the polynucleotides and/or target nucleic acid are not each bound to single stranded binding proteins. In some embodiments, the annealing of polynucleotides and target nucleic acid is faster when the sample is contacted with the strand annealing protein and the nucleic acid probe and target nucleic acid are each bound to a single stranded binding proteins, as compared to when the sample is not contacted with the strand annealing protein and the nucleic acid probe and target nucleic acid are not each bound to single stranded binding proteins.

In some embodiments, the annealing of the polynucleotides (e.g., hybridization of the first nucleic acid sequence to the third nucleic acid sequence and/or hybridization of the second nucleic acid sequence to the third nucleic acid sequence) is at least about 2-fold, at least about 5-fold, at least about 10-fold, at least about 15-fold, at least about 20-fold, or at least about 25-fold faster in the presence of the single strand annealing protein compared to annealing of the same polynucleotides under the same conditions in the absence of the single strand annealing protein. In some embodiments, the annealing of the polynucleotides (e.g., hybridization of the first nucleic acid sequence to the third nucleic acid sequence and/or hybridization of the second nucleic acid sequence to the third nucleic acid sequence) is between about 2-fold and about 5-fold faster, between about 2-fold and about 10-fold faster, between about 5-fold and about 15-fold faster, or between about 10-fold and about 25-fold faster in the presence of the single strand annealing protein compared to annealing of the same polynucleotides under the same conditions in the absence of the single strand annealing protein.

In some embodiments, the SSAP promotes ligation and/or binding/hybridization. In some embodiments, the SSAP promotes the ligation of the first nucleic acid sequence and/or the second nucleic acid sequence using the third nucleic acid sequence (e.g., a target nucleic acid) as a template. In some embodiments, ligation efficiency is increased by at least 10%, at least 20%, at least 30%, at least 40%, at least 50%, at least 60%, at least 70%, at least 80%, at least 90%, at least 100%, at least 200%, or at least 300% in the presence of the single strand annealing protein compared to annealing of the same polynucleotides under the same conditions in the absence of the single strand annealing protein. In some embodiments, the single strand annealing protein is DdrB or an engineered variant or derivative thereof (e.g., a polypeptide having at least 50%, at least 60%, at least 70%, at least 80%, at least 85%, at least 90%, or at least 95% amino acid sequence identity). DdrB is a single strand annealing protein derived from Deinococcus bacteria. The DdrB can be a DdrB from any species of Deinococcus, or an engineered variant or derivative thereof. In some embodiments, the DdrB is Deinococcus radiodurans (D. radiodurans) DdrB, D. geothermalis DdrB, or D. deserti DdrB, or an engineered variant thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to the sequence of any of SEQ ID NOs: 3-5. In some embodiments, the single strand annealing protein comprises the amino acid sequence of any of SEQ ID NOs: 3-5.

In some embodiments, the single strand annealing protein promotes the hybridization of the first nucleic acid sequence and/or the second nucleic acid sequence to the third nucleic acid sequence (e.g., in a target nucleic acid such as a target RNA). In some embodiments, the single strand annealing protein ligation between the first nucleic acid sequence and the second nucleic acid sequence using the third nucleic acid sequence (e.g., in a target nucleic acid such as a target RNA) as a template.

In some embodiments, the level of annealing of the polynucleotides and target nucleic acid is greater when the sample is contacted with the strand annealing protein as compared to when it is not contacted with the strand annealing protein. In some cases, the reaction for single stranded protein mediated hybridization can be optimized for the desired level of binding/hybridization (e.g., by adjusting the concentration of the probes, concentration of single stranded binding proteins, concentration of strand annealing proteins, and/or level of salt).

In some embodiments, the single strand annealing protein is DdrB or an engineered variant or derivative thereof (e.g., a polypeptide having at least 50%, at least 60%, at least 70%, at least 80%, at least 85%, at least 90%, or at least 95% amino acid sequence identity). DdrB is a single strand annealing protein derived from Deinococcus bacteria. The DdrB can be a DdrB from any species of Deinococcus, or an engineered variant or derivative thereof. In some embodiments, the DdrB is Deinococcus radiodurans (D. radiodurans) DdrB, D. geothermalis DdrB, or D. deserti DdrB, or an engineered variant thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to the sequence of any of SEQ ID NOs: 3-5. In some embodiments, the single strand annealing protein comprises the amino acid sequence of any of SEQ ID NOs: 3-5.

In some embodiments of any of the methods disclosed herein, the single strand annealing protein is selected from the group consisting of Rad52, Redβ, Erf, RecT, Sak, ICP8, RecT, DdrB, RecO, UvsY, and Hfq, and variants or derivatives thereof. In some embodiments of the methods disclosed herein, the strand annealing protein may be derived from a bacteriophage (e.g., T4 phage UvsY), bacteria (e.g., Escherichia. coli or Deinococcus RecT or RecO), archaea (e.g., S. solfataricus RadB), or eukaryotes (e.g., S. cerevisiae, mouse, or human Rad52). In some cases, the strand annealing protein can be a functional homolog or fragment thereof of any of the provided exemplary proteins. In some embodiments, the single strand annealing protein is D. radiodurans DdrB. The structure of the DdrB-ssDNA complex shares several key features with other DNA and RNA single strand annealing proteins Rad52, Redβ, Erf, RecT, Sak, ICP8, and Hfq, despite these proteins not being true homologs. In some embodiments, the single strand annealing protein is E. coli RecT. In some cases, the strand annealing protein is expressed and purified according to standard laboratory techniques. In some embodiments, the single strand annealing protein comprises a purification tag such as a polyhistidine tag. Sequences of various exemplary single strand annealing proteins are provided in Table 1 below.

TABLE 1
Exemplary single strand annealing proteins

| | | SEQ |
|---|---|---|
| Description | Sequence | ID NO: |
| RecT (from | MTKQPPIAKADLQKTQGNRAPAAVKNS | 1 |
| Escherichia | DVISFINQPSMKEQLAAALPRHMTAER | |
| coli) | MIRIATTEIRKVPALGNCDTMSFVSAI | |
| | VQCSQLGLEPGSALGHAYLLPFGNKNE | |
| | KSGKKNVQLIIGYRGMIDLARRSGQIA | |
| | SLSARVVREGDEFSFEFGLDEKLIHRP | |
| | GENEDAPVTHVYAVARLKDGGTQFEVM | |
| | TRKQIELVRSLSKAGNNGPWVTHWEEM | |
| | AKKTAIRRLFKYLPVSIEIQRAVSMDE | |
| | KEPLTIDPADSSVLTGEYSVIDNSEE | |
| Lambda | STALATLAGKLAERVGMDSVDPQELIT | 2 |
| phage Redß | TLRQTAFKGDASDAQFIALLIVANQYG | |
| | LNPWTKEIYAFPDKQNGIVPVVGVDGW | |
| | SRIINENQQFDGMDFEQDNESCTCRIY | |
| | RKDRNHPICVTEWMDECRREPFKTREG | |
| | REITGPWQSHPKRMLRHKAMIQCARLA | |
| | FGFAGIYDKDEAERIVENTAYTAERQP | |
| | ERDITPVNDETMQEINTLLIALDKTWD | |
| | DDLLPLCSQIFRRDIRASSELTQAEAV | |
| | KALGFLKQKAAEQKVAA | |
| DdrB | MLQIEFITDLGARVTVNVEHESRLLDV | 3 |
| (GenBank: | QRHYGRLGWTSGEIPSGGYQFPIENEA | |
| QIP28318.1 | DFDWSLIGARKWKSPEGEELVIHRGHA | |
| from | YRRRELEAVDSRKLKLPAAIKYSRGAK | |
| Deinococcus | VSDPQHVREKADGDIEYVSLAIFRGGK | |
| radiodurans) | RQERYAVPGGAAGNGQGRPAPQGQPAQ | |
| | ARPQATAARPAARPPVQPGQEEETPF | |
| DdrB (Dgeo_ | MLHIEFITDLGAKVTVDVESADKLLDV | 4 |
| 0070) from | QRQYGRLGWTSGEVPVGGYQFPLENEP | |
| Deinococcus | DFDWSLIGARKWINPEGEEMILHRGHA | |
| geothermalis | YRRRELEAVDSRKMKLPAAVKYSRGAK | |
| | NTDPEHVREKADGEFEYVTLAIFRGGK | |
| | RQERYAVPGSNRPQAGAPARSAATRAQ | |
| | GARPGAVAVQDEETPF | |
| DdrB (NCBI | MLHIEFLTDLGAKVTVDVEHENQLLDV | 5 |
| | QRHYGRLGWTSGAVPTGGYQFPLDNEP | |
| Reference | DFDWSLIGARKWTSPDGEELVIHKGHA | |
| Sequence: | YRRRELEAVDSRKMKLPAAVKFSRGAK | |
| WP_012692241.1) | STDPEHLREKADGEFEYVTLAIFRGGK | |
| from | RQERYAVPGGARPAAQSARPAPARAQA | |
| Deinococcus | AARPAPVAVVDEETPF | |
| deserti | | |

In some embodiments, a first single strand annealing protein promotes annealing of the first single-stranded nucleic acid sequence and a first single-stranded hybridization region to form a duplex. In some embodiments, a second strand annealing protein promotes annealing of a second single-stranded nucleic acid sequence and a second single-stranded hybridization region to form a duplex. In some embodiments, the first single strand annealing protein and the second single strand annealing protein are the same. In some embodiments, the first single strand annealing protein and the second single strand annealing protein are different.

In some aspects, the method disclosed herein comprises ligating a first nucleic acid sequence and a second nucleic acid sequence using a third nucleic acid sequence a template to generate a ligated oligonucleotide comprising the first nucleic acid sequence and the second nucleic acid sequence. In some aspects, the method comprises ligating the first sequence of the probe or probe set (e.g., circularizable probe) to a second sequence hybridized to a second hybridization region of the target nucleic acid in the presence of a single strand annealing protein bound to the first sequence and/or the second sequence, wherein the second hybridization region is adjacent to the first hybridization region in the target nucleic acid, and wherein the second sequence is a part of the probe or probe. In some embodiments, the method comprises detecting the ligated oligonucleotide, probe or product thereof comprising the first sequence and second sequence or a product thereof.

In some embodiments, the single strand annealing protein promotes ligation and/or binding of the first nucleic acid sequence and the second nucleic acid sequence using the third nucleic acid sequence as a template. In some aspects, the ligation efficiency is increased by at least 10%, at least 20%, at least 30%, at least 40%, at least 50%, at least 60%, at least 70%, at least 80%, at least 90%, or at least 100% compared to the ligation efficiency for the same nucleic acid sequences under the same conditions but in the absence of the single strand annealing protein. In some aspects, the ligation efficiency is increased by at least 2-fold, at least 2.5-fold, at least 3-fold, or at least 5-fold compared to the ligation efficiency for the same nucleic acid sequences under the same conditions but in the absence of the single strand annealing protein. In some embodiments, hybridization of the first nucleic acid sequence to the first hybridization region is increased by at least 10%, at least 20%, at least 30%, at least 40%, at least 50%, at least 60%, at least 70%, at least 80%, at least 90%, or at least 100% compared to the level of hybridization for the same nucleic acid sequences under the same conditions but in the absence of the single strand annealing protein. In some aspects, the hybridization is increased by at least 2-fold, at least 2.5-fold, at least 3-fold, or at least 5-fold compared to the hybridization for the same nucleic acid sequences under the same conditions but in the absence of the single strand annealing protein. In some embodiments, the ligation efficiency for ligating the first nucleic acid sequence to the second nucleic acid sequence in the presence of the single strand annealing protein is at least 70%, at least 75%, or at least 80%.

In some embodiments, the third nucleic acid sequence is RNA and the first nucleic acid sequence and/or second nucleic acid sequence comprises DNA. In some embodiments, the third nucleic acid is an RNA, and the method comprises providing the single strand annealing protein at a concentration of at least about 10 nM, at least about 15 nM, at least about 30 nM, at least about 100 nM, or at least about 300 nM. In some embodiments, the method comprises providing the single strand annealing protein at a concentration of no more than about 500 nM, no more than about 400 nM, no more than about 300 nM, no more than about 250 nM, no more than about 200 nM, no more than about 100 nM, no more than about 50 nM, no more than about 30 nM, no more than about 15 nM, or no more than about 10 nM. In some embodiments, the concentration of the single strand annealing protein is less than about 500 nM (e.g., between about 10 nM and about 500 nM). In some embodiments, the single strand annealing protein is selected from the group consisting of Rad52, Redβ, Erf, RecT, Sak, ICP8, RecT, DdrB, RecO, UvsY, Hfq, and variants or derivatives thereof. In some embodiments, the single strand annealing protein is DdrB or an engineered variant or derivative thereof (e.g., a polypeptide having at least 50%, at least 60%, at least 70%, at least 80%, at least 85%, at least 90%, or at least 95% amino acid sequence identity). DdrB is a single strand annealing protein derived from Deinococcus bacteria. The DdrB can be a DdrB from any species of Deinococcus, or an engineered variant or derivative thereof. In some embodiments, the DdrB is Deinococcus radiodurans (D. radiodurans) DdrB, D. geothermalis DdrB, or D. deserti DdrB, or an engineered variant thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to the sequence of any of SEQ ID NOs: 3-5. In some embodiments, the single strand annealing protein comprises the amino acid sequence of any of SEQ ID NOs: 3-5.

In some embodiments, the third nucleic acid sequence is DNA, and the concentration of single strand annealing protein used is at least 10 nM, at least 20 nM, at least 30 nM, at least 50 nM, at least 100 nM, at least 300 nM, at least 500 nM, at least 700 nM, at least 1000 nM. In some embodiments, the concentration of SSAP used is at least 100 nM, at least 150 nM, at least 200 nM, at least 250 nM, at least 300 nM, at least 350 nM, at least 400 nM, at least 450 nM, at least 500 nM, at least 550 nM, at least 600 nM, at least 650 nM, at least 700 nM, at least 750 nM, at least 800 nM, at least 850 nM, at least 900 nM, at least 950 nM, or at least 1000 nM. In some embodiments, the method comprises providing the single strand annealing protein at a concentration of no more than about 1000 nM, no more than about 900 nM, no more than about 800 nM, no more than about 700 nM, no more than about 600 nM, no more than about 500 nM, no more than about 400 nM, no more than about 300 nM, no more than about 200 nM, no more than about 100 nM, no more than about 50 nM, or no more than about 20 nM. In some embodiments, the concentration of the single strand annealing protein is less than about 500 nM (e.g., between about 10 nM and about 500 nM). In some embodiments, the single strand annealing protein is selected from the group consisting of Rad52, Redβ, Erf, RecT, Sak, ICP8, RecT, DdrB, RecO, UvsY, Hfq, and variants or derivatives thereof. In some embodiments, the single strand annealing protein is DdrB or an engineered variant or derivative thereof (e.g., a polypeptide having at least 50%, at least 60%, at least 70%, at least 80%, at least 85%, at least 90%, or at least 95% amino acid sequence identity). DdrB is a single strand annealing protein derived from Deinococcus bacteria. The DdrB can be a DdrB from any species of Deinococcus, or an engineered variant or derivative thereof. In some embodiments, the DdrB is Deinococcus radiodurans (D. radiodurans) DdrB, D. geothermalis DdrB, or D. deserti DdrB, or an engineered variant thereof. In some embodiments, the single strand annealing protein comprises an amino acid sequence having at least about 80%, at least about 85%, at least about 90%, at least about 95%, at least about 96%, at least about 97%, at least about 98%, or at least about 99% amino acid sequence identity to the sequence of any of SEQ ID NOs: 3-5. In some embodiments, the single strand annealing protein comprises the amino acid sequence of any of SEQ ID NOs: 3-5.

In any one of the embodiments herein, the ligation can comprise using a ligase having an RNA-templated DNA ligase activity and/or an RNA-templated RNA ligase activity. In some embodiments, the enzymatic ligation involves use of a ligase (e.g., an RNA ligase or a DNA ligase). In some embodiments, the ligase is a DNA ligase. In some embodiments, the ligase is a RNA ligase. Ligases comprise ATP-dependent double-strand polynucleotide ligases, NAD-i-dependent double-strand DNA or RNA ligases and single-strand polynucleotide ligases, for example any one of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In any one of the embodiments herein, the ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any one of the embodiments herein, the ligation can comprise using a PBCV-1 DNA ligase or variant or derivative thereof and/or a T4 RNA ligase 2 (T4 Rnl2) or variant or derivative thereof. In some embodiments, the ligase is a T4 RNA ligase. In some embodiments, the ligase comprises a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity. In some embodiments, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tm) of the DNA strands. High-fidelity ligation can be achieved through a combination of the intrinsic selectivity of the ligase active site and balanced conditions to reduce the incidence of annealed mismatched dsDNA.

In some embodiments, the ligation herein is a direct ligation. In some embodiments, the ligation herein is an indirect ligation. In some embodiments, a direct ligation occurs between ends of the polynucleotides hybridize immediately adjacently to one another to form a substrate for a ligase enzyme resulting in their ligation to each other (intramolecular ligation). In some embodiments, an indirect ligation is performed when the ends of the polynucleotides hybridize non-adjacently to one another, e.g., separated by one or more intervening nucleotides or gaps. In some embodiments, said ends are not ligated directly to each other, but instead occurs either via the intermediacy of one or more intervening (so-called gap or gap-filling (oligo)nucleotides) or by the extension of the 3′ end of a probe to fill the “gap” corresponding to said intervening nucleotides (intermolecular ligation). In some cases, the gap of one or more nucleotides between the hybridized ends of the polynucleotides may be filled by one or more gap (oligo)nucleotide(s) which are complementary to a splint, circularizable probe or probe set (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

Various embodiments of the methods disclosed herein for single strand annealing protein-assisted nucleic acid sequence annealing (e.g., hybridization) and/or ligation in situ in a biological sample are described in Section A below. Further exemplary embodiments of the methods disclosed herein for generating spatial arrays or barcoded molecules (e.g., barcoded beads) are described in further detail in Sections B and C below. In any of the embodiments described in Sections A-C, the single strand annealing protein can be any of the single strand annealing proteins described above.

### A. Protein-Mediated Strand Annealing

Disclosed herein in some aspects, is a method for nucleic acid ligation, comprising: a) providing a first nucleic acid strand, a second nucleic acid strand, and a third nucleic acid strand; wherein the first nucleic acid strand comprises a first sequence that hybridizes to a first hybridization region of the third nucleic acid strand in the biological sample, and the second nucleic acid strand comprises a second sequence that hybridizes to a second hybridization region of the third nucleic acid strand wherein the first nucleic acid strand, second nucleic acid strand and/or the third nucleic acid strand are/is bound to a single strand annealing protein; and b) ligating the first nucleic acid strand and second nucleic acid strand using the third nucleic acid strand as template to generate a ligated oligonucleotide comprising the first sequence and the second sequence. The biological sample can be any of the samples described in Subsection c below. In some embodiments, the biological sample is a tissue sample.

In some aspects, provided herein is a method for nucleic acid ligation, comprising: a) contacting a biological sample with a probe comprising a first nucleic acid sequence, wherein the probe hybridizes to a first hybridization region of a target nucleic acid; b) ligating the first sequence of the probe or probe set to a second nucleic acid sequence hybridized to a second hybridization region of the target nucleic acid in the presence of a single strand annealing protein bound to the first sequence and/or the second sequence, thereby generating a ligated probe. In some embodiments, the second hybridization region is adjacent to the first hybridization region in the target nucleic acid, and the ligation is not preceded by gap filling. In some cases wherein the target nucleic acid comprises DNA, the first hybridization region is separated from the second hybridization region by 1, 2, 3, 4, 5, 6, 10, or more nucleotides, and the ligation is preceded by gap filling using a polymerase. In some embodiments, the second sequence is in a second probe or in the same probe (e.g., at the opposite end of the same probe, as in a circularizable probe). In some embodiments, the method comprises detecting the ligated probe or a product thereof. In some embodiments, detecting the ligated probe or a product thereof serves as a proxy for detecting the target nucleic acid (e.g., a nucleic acid analyte or a nucleic acid associated with an analyte in the biological sample).

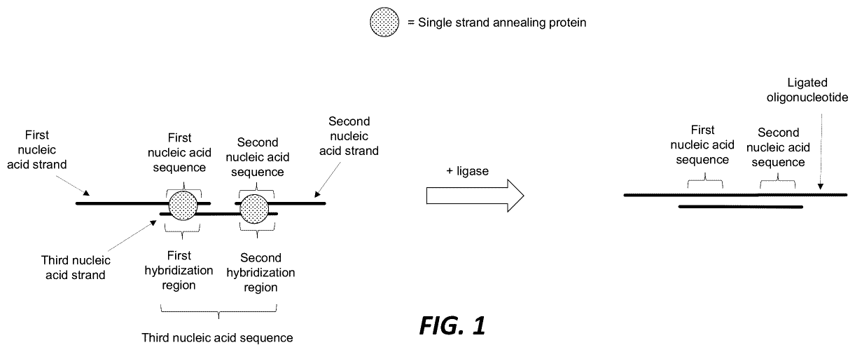

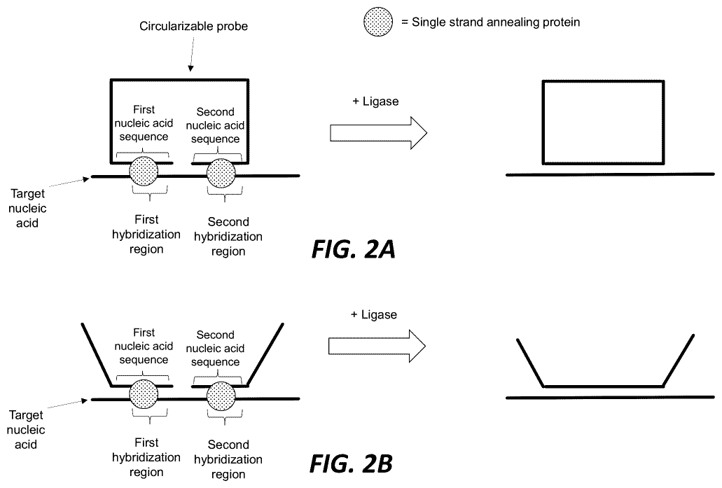

In some embodiments, the single strand annealing proteins bound to the polynucleotides facilitate the annealing of the polynucleotides to the target nucleic acid. For example, the single strand annealing protein facilitates the annealing of the first and second sequences of the first and second nucleic acid strands to the first and second hybridization regions of the target nucleic acid, respectively (e.g., as illustrated in FIG. 1). In some embodiments, first and second nucleic acid sequences are at opposite ends of a circularizable probe, and the single strand annealing protein facilitates the annealing of the first and second nucleic acid sequences of the circularizable probe (e.g., sequences at the 3′ and 5′ ends of the circularizable probe) to the first and second hybridization regions of the target nucleic acid, respectively (e.g., as shown in FIG. 2A). In some embodiments, the single strand annealing protein facilitates the annealing of the first and second sequences of the first and second probes, respectively (e.g., as illustrated in FIG. 2B). In some embodiments, the single strand annealing protein increases the efficiency of ligation of the probe or probes. In some embodiments, the single strand annealing protein increases the efficiency of ligation of the probe or probes in situ in the biological sample (e.g., in a tissue sample or matrix-embedded sample).

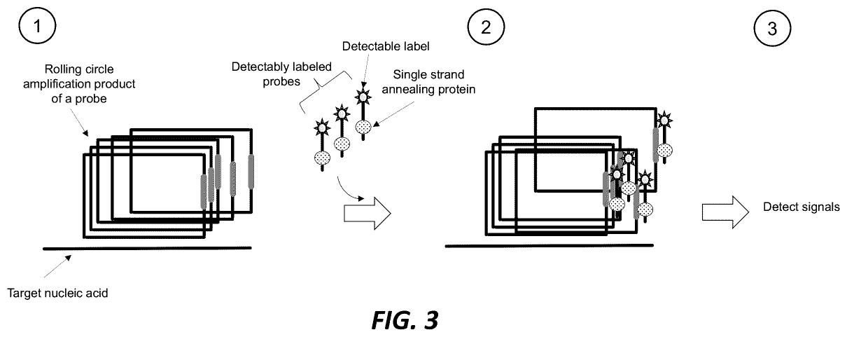

In some embodiments, provided herein is a method for analyzing a biological sample, comprising contacting a target nucleic acid in the biological sample with a probe, wherein the probe is bound to a single strand annealing protein and the probe comprises a recognition sequence that hybridizes to a target sequence in the target nucleic acid, and detecting a signal associated with the probe, thereby detecting the target nucleic acid in the biological sample. In some embodiments, the single strand annealing protein facilitates the annealing of probes to complementary target sequences in a target nucleic acid (e.g., a target sequence in a rolling circle amplification product, as illustrated in FIG. 3). In some embodiments, the probe is a detectably labeled probe such as a fluorescently labeled probe. In some embodiments, the probe comprises a binding site for a detectably labeled oligonucleotide associated with the signal, or for an intermediate probe that binds directly or indirectly to a detectably labeled oligonucleotide, and the method comprises contacting the biological sample with the detectably labeled oligonucleotide and/or intermediate probe, thereby associating the signal with the probe hybridized to the target nucleic acid. In some embodiments, the target nucleic acid is a DNA concatemer comprising multiple copies of the target sequence.

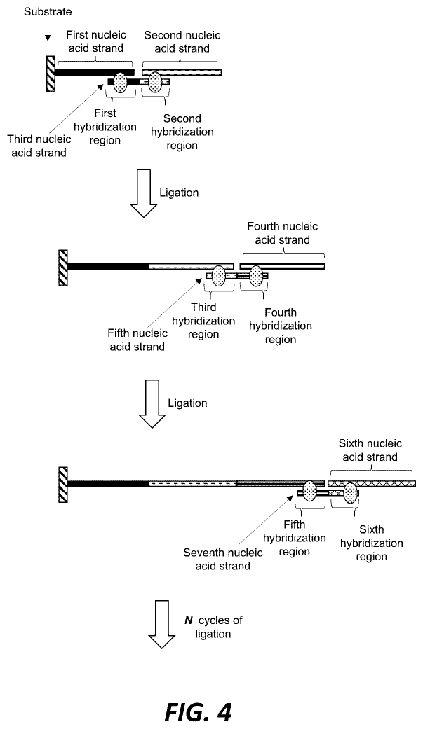

FIGS. 1, 2A-2B, and 3 depict schematics of exemplary polynucleotides. FIG. 1 depicts three polynucleotides: a first nucleic acid strand, a second nucleic acid strand, and a third nucleic acid strand. The third nucleic acid strand may be a target nucleic acid such as any of the target nucleic acids described herein. Each of the polynucleotides (e.g., nucleic acid strands) includes at least one sequence, such as regions that are complementary or sufficiently complementary to a different nucleic acid strand sequence (for example, to the target nucleic acid, such as an mRNA, or to another hybridization region present in one of the polynucleotides) to form complexes via Watson-Crick base pairing. The first nucleic acid strand comprises a first sequence that is complementary or sufficiently complementary to a first hybridization region on the third nucleic acid strand (e.g., target nucleic acid). The second nucleic acid strand comprises a second sequence that is complementary or sufficiently complementary to a second hybridization region on the third nucleic acid strand (e.g., target nucleic acid). Any one of the polynucleotides may also be bound to a single strand annealing protein. In some cases, one or more of the nucleic acid strands is bound to a single strand annealing protein before contacting the nucleic acid strand with another nucleic acid strand (e.g., before contacting a biological sample with a first and/or second nucleic acid strand, or before contacting an immobilized first nucleic acid strand with a first and/or third nucleic acid strand (e.g., in the method illustrated in FIG. 4).

FIG. 2A depicts an exemplary circularizable polynucleotide (e.g., a circularizable probe such as a padlock probe). The circularizable probe can be any probe that hybridizes to one or more other nucleic acid strands such that the ends of the probe are ligatable to each other, with or without gap filling prior to ligation. In an example shown in FIG. 2A, the circularizable probe comprises a first sequence that is complementary to a first hybridization region on the target nucleic acid (e.g., an mRNA molecule). The circularizable probe also comprises a second sequence that is complementary to a second hybridization region on the target nucleic acid (e.g., an mRNA molecule). The first sequence may be at the 3′ end and the second sequence may be at a 5′ end of a circularizable probe or probe set. Alternatively, the first sequence may be at the 5′ end and the second sequence may be at a 3′ end of a circularizable probe or probe set. FIG. 2B depicts an exemplary probe set (e.g., a first probe and a second probe). In an example shown in FIG. 2B, the first probe comprises a first sequence that is complementary to a first hybridization region on the target nucleic acid (e.g., an mRNA molecule); the second probe comprises a second sequence that is complementary to a second hybridization region on the target nucleic acid (e.g., an mRNA molecule). The 3′ end of the first probe and the 5′ end of the second probe of the probe set can be ligated. As shown in FIG. 2B, the first sequence of the first probe and the second sequence of the second probe are bound to a single strand annealing protein. The single strand annealing protein facilitates annealing and improves the ligation efficiency and binding of the first and second sequences of the circularizable probe or probe set to the target nucleic acid.

FIG. 3 depicts an exemplary polynucleotide and single strand annealing protein complexes (e.g., probes bound to single strand annealing proteins) that are used to detect the presence of a target nucleic acid (e.g., rolling circle amplification product (RCP) of a probe bound to a target nucleic acid). In some cases, as shown in FIG. 3, detectably labeled probes comprising a sequence that is complementary or sufficiently complementary to a target sequence in an RCP are hybridized to the RCP in the presence of single strand annealing proteins. In some cases, the detectably labeled probes include detectable labels, such as fluorescent labels that emit signals and facilitate the visualization of the hybridization complexes. In some embodiments, the probes themselves do not comprise detectable labels, but comprise a binding site for hybridization of a detectably labeled oligonucleotide or an intermediate probe. In some embodiments, the sequence of the binding site corresponds to a detectably labeled oligonucleotide or intermediate probe that is associated with a particular signal or with the absence of a signal. In some embodiments, the binding site for the detectably labeled oligonucleotide or intermediate probe is in an overhang region of the probe that does not hybridize to the target nucleic acid. In some embodiments, the overhang region and/or the detectably labeled oligonucleotide or intermediate probe is bound by a single strand annealing protein. In some embodiments, the single strand annealing protein increases the efficiency of hybridization of the probes to their corresponding target sequences in the target nucleic acid(s). In some embodiments, the single strand annealing protein increases the specificity of hybridization of the probes to their corresponding target sequences in the target nucleic acid(s).

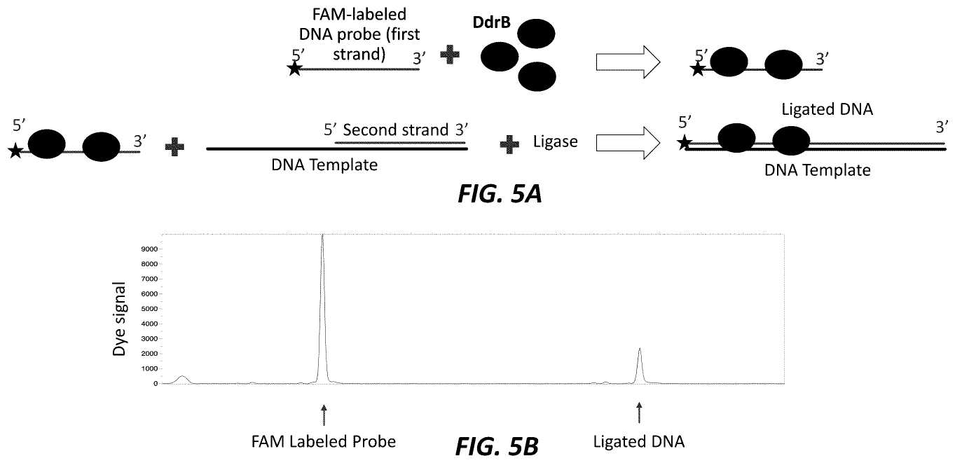

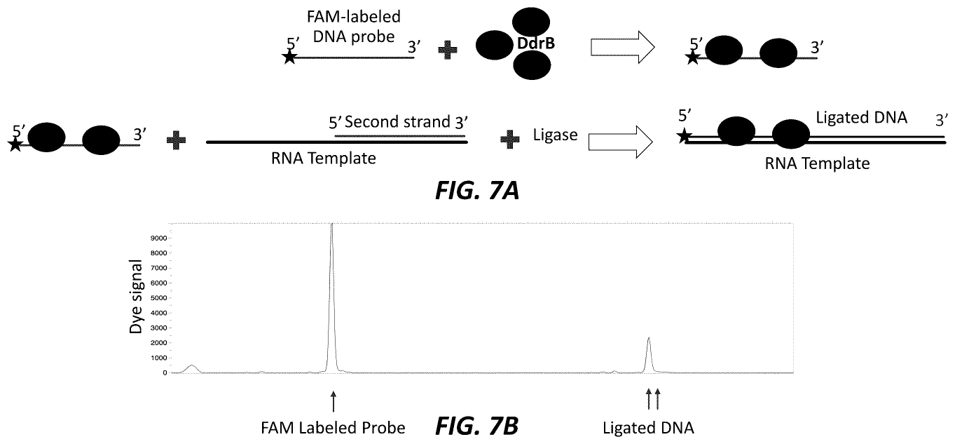

In some embodiments, the first nucleic acid sequence is in a probe, and the method comprises contacting a biological sample with the probe, wherein the third nucleic acid sequence is in a target nucleic acid in the biological sample, wherein the second nucleic acid sequence is in the same probe or is in a second probe hybridized to the target nucleic acid (e.g., as shown in FIGS. 5A and 7A), wherein the ligating is performed in the biological sample, and wherein the ligated oligonucleotide is a ligated probe comprising the first nucleic acid sequence and the second nucleic acid sequence.

### a) Probes and Target Sequences

In some aspects, the methods provided herein comprise contacting a biological sample with are polynucleotides (e.g., nucleic acid probes and/or probe sets, nucleic acid molecules, nucleic acid strands) that are introduced into a cell or used to otherwise contact a biological sample such as a tissue sample. Provided are polynucleotides (e.g., first and second nucleic acid strands) comprising at least one sequence capable of hybridizing to target regions (e.g., hybridization regions) in a third nucleic acid sequence (e.g., in a target nucleic acid). Also provided are probes and/or probe sets (e.g., circularizable probe or first and second probes) comprising at least two sequences (e.g., first and second sequence) capable of hybridizing to hybridization regions (e.g., first and second hybridization regions, respectively) in a target nucleic acid. In some aspects, the probes provided herein are bound to single strand annealing proteins. In some aspects, the single strand annealing proteins promote ligation and/or binding of the sequences (e.g., first sequence and/or the second sequence) to the target nucleic acids.

In some aspects, the probe or probe set (e.g., a circularizable probe or a first and second probe) comprise a first sequence capable of hybridizing to a first hybridization region in a target nucleic acid and a second sequence capable of hybridizing to a second hybridization region in a target nucleic acid in the biological sample. In some embodiments, the first and second sequences are the same probe (e.g., at either end of a circularizable probe). In embodiments, the first sequence is located in a first probe and the second sequence is located in a second probe. In some embodiments, the first probe is ligated to the second probe using the target nucleic acid as template to generate a ligated probe. In some embodiments, the 3′ and a 5′ ends of the probe or probe sets are ligated together to form a circular probe.

The probes may comprise any one of a variety of entities that can hybridize to a nucleic acid, typically by Watson-Crick base pairing, such as DNA, RNA, LNA, PNA, etc. The probes may be able to bind to a specific target nucleic acid (e.g., an mRNA, or other nucleic acids as discussed herein). In some embodiments, the probes are detected using a detectable label, and/or by using secondary (intermediate) nucleic acid probes able to bind to the probes and to one or more detectably labeled oligonucleotides. In some embodiments, the probes are compatible with one or more biological and/or chemical reactions. For instance, a probe or ligated probe disclosed herein can serve as a template or primer for a polymerase, a template or substrate for a ligase, and/or a substrate for a nuclease (e.g., endonuclease for cleavage).

In some embodiments, the first nucleic acid sequence that hybridizes to the first hybridization region is at least about 4, at least about 6, at least about 8, at least about 10, at least about 15, at least about 20, at least about 25, at least about 30, at least about 35, or at least about 40 nucleotides in length. In some embodiments, the first nucleic acid sequence that hybridizes to the first hybridization region is between about 4 and 50, between about 4 and about 40, between about 4 and about 30, between about 8 and about 50, between about 8 and about 40, between about 10 and about 30, or between about 10 and about 25 nucleotides in length. In some embodiments, the first hybridization region is at least about 4, at least about 6, at least about 8, at least about 10, at least about 15, at least about 20, at least about 25, at least about 30, at least about 35, or at least about 40 nucleotides in length. In some embodiments, the first hybridization region is between about 4 and between about 4 and about 40, between about 4 and about 30, between about 8 and about 50, between about 8 and about 40, between about 10 and about 30, or between about 10 and about 25 nucleotides in length.

In some embodiments, the second nucleic acid sequence that hybridizes to the second hybridization region is at least about 4, at least about 6, at least about 8, at least about 10, at least about 15, at least about 20, at least about 25, at least about 30, at least about 35, or at least about 40 nucleotides in length. In some embodiments, the second nucleic acid sequence that hybridizes to the second hybridization region is between about 4 and 50, between about 4 and about between about 4 and about 30, between about 8 and about 50, between about 8 and about 40, between about 10 and about 30, or between about 10 and about 25 nucleotides in length. In some embodiments, the second hybridization region is at least about 4, at least about 6, at least about 8, at least about 10, at least about 15, at least about 20, at least about 25, at least about 30, at least about 35, or at least about 40 nucleotides in length. In some embodiments, the second hybridization region is between about 4 and 50, between about 4 and about 40, between about 4 and about 30, between about 8 and about 50, between about 8 and about 40, between about 10 and about 30, or between about 10 and about 25 nucleotides in length.

In some embodiments, provided herein are methods and compositions for single strand annealing protein-assisted probe annealing and/or ligation for analyzing endogenous analytes (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof. In some embodiments, a third nucleic strand described herein is a target nucleic acid. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed. A target sequence for a probe disclosed herein may be comprised in any analyte disclose herein, including an endogenous analyte (e.g., a viral or cellular nucleic acid), a labelling agent, or a product of an endogenous analyte and/or a labelling agent.

A target nucleic acid disclosed herein can be any polynucleotide (e.g., DNA molecule; RNA molecule, modified nucleic acid, etc.) for assessment in accordance with the provided embodiments, such as a polynucleotide present in a cell. In some embodiments, the target nucleic acid is a coding RNA (e.g., mRNA). The target may, in some embodiments, be a single RNA molecule. In other embodiments, the target may be at least one RNA molecule, e.g., a group of 2, 3, 4, 5, 6 or more RNA molecules. These RNA molecules may differ in molecule type, and/or may differ in sequence. In some embodiments, the target nucleic acid is, for example, a non-coding RNA (e.g., tRNA, rRNA, microRNA (miRNA), mature miRNA or immature miRNA). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, e.g., have a differing abundance, within a cell population, wherein the methods of the present disclosure allow profiling and comparison of the expression levels of nucleic acids, comprising but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like. In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA.

In some embodiments, the target nucleic acid comprises RNA. In some embodiments, the target nucleic acid comprises mRNA. In some embodiments, the probe comprises DNA. In some embodiments, the target nucleic acid is RNA and the probe or probe set comprises DNA. In some aspects, the DNA probes hybridize to the target RNA and are ligated to from a ligated oligonucleotide or ligated probe using the RNA as template in the presence of a single strand annealing protein. In some embodiments, the single strand annealing protein binds to one or more of the DNA probes. In some embodiments, the single strand annealing protein binds to the RNA. In some embodiments, the probe or probe set (e.g., the first nucleic acid sequence or the second nucleic acid sequence in the probe or probe set) comprises a ribonucleotide (e.g., the probe or probe set is a DNA/RNA chimera comprising at least one ribonucleotide). In some embodiments, the ribonucleotide is a 3′ end ribonucleotide of the probe or probe set. In some embodiments, the 5′ end nucleotide of the probe or probe set is DNA. In some embodiments, the probe or probe set (e.g., the first nucleic acid sequence or the second nucleic acid sequence in the probe or probe set) comprises no more than four consecutive ribonucleotides. In some embodiments, the probe or probe set does not comprise RNA.

In some aspects, the methods provided herein are used to analyze a target nucleic acid, e.g., a messenger RNA molecule. In some embodiments, the target nucleic acid is an endogenous nucleic acid present in a biological sample. In some embodiments, the target nucleic acid is present in a cell in a tissue, for instance in a tissue sample or tissue section. In some embodiments, the tissue sample is an intact tissue sample or a non-homogenized tissue sample. In some embodiments, the tissue sample is a fresh tissue sample. In some embodiments, the tissue has previously been processed, e.g., fixed, embedded, frozen, or permeabilized using any one of the steps and/or protocols described in Subsection c below. In some embodiments, the target nucleic acid is an exogenous nucleic acid contacted with a biological sample. In some embodiments, the target nucleic acid is present in a single cell, for instance in a dissociated single cell sample.

In some aspects, the provided embodiments can be employed for in situ detection of a target nucleic acid in a cell, e.g., in cells of a biological sample or a sample derived from a biological sample, such as a tissue section on a solid support, such as on a transparent slide.

The specific probe or probe set design can vary. Exemplary circularizable probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, or a PLISH (Proximity Ligation in situ hybridization) probe set. For example, FIG. 2A depicts a circularizable probe comprising a first sequence and a second sequence that hybridize to a first hybridization region and a second hybridization region of a target nucleic acid, respectively. FIG. 2A depicts a probe set comprising a first sequence and a second sequence that hybridize to a first hybridization region and a second hybridization region of a target nucleic acid, respectively. In some embodiments, the first and second sequences are part of the circularizable probe. In some embodiments, the first sequence is at the 5′ end and the second sequence is at the 3′ end of the probe. In some embodiments, the first sequence is at the 3′ end and the second sequence is at the 5′ end of the probe. In some embodiments, the first sequence is part of a first probe and the second sequences is part of a second probe. In some embodiments, the 3′ end of the first sequence is ligated to the 5′ end of the second sequence. In some embodiments, the 5′ end of the first sequence is ligated to the 3′ end of the second sequence. In some embodiments, the ligated oligonucleotide is a circular probe. In some embodiments, a single stranded binding protein binds to the first sequence and/or the second sequence of the circularizable probe or probe set (e.g., as shown in FIGS. 2A-2B). In some embodiments, the single stranded binding protein promotes ligation and/or binding of the first sequence and/or the second sequence to the target nucleic acid. In some embodiments, the circularizable probe and the single strand annealing protein are contacted with the biological sample simultaneously or sequentially. The 5′ and 3′ ends of the circularizable probes or linear first or second probe can optionally be extended by a polymerase, using the target nucleic acid as a template and ligated using a ligase (such as a T4 DNA ligase, a PBCV-1 DNA ligase, or a T4 RNA ligase 2 (T4 Rnl2)). The processed probe can be ligated to circularize the circularizable probe. The ligated probe can then be amplified to a rolling circle amplification product (RCA) and then detected. In some instances, a processed probe set comprising a linear first and a second probe can be ligated together to form a linear ligated probe product (e.g., ligated oligonucleotide).

In some embodiments, a circularized probe or probe set (e.g., a DNA probe that directly binds to an RNA target) is amplified through rolling circle amplification (RCA). In some embodiments a circularizable probe or probe set is ligated, circularized, and amplified. In some embodiments the circularizable probe or probe set is ligated using a target nucleic acid as a template, optionally wherein the target nucleic acid is an RNA. In some embodiments, a circular probe can be generated by ligating the 3′ and 5′ ends of a circularizable probe (e.g., as shown in FIG. 2A).

In some embodiments, a probe or probe set (e.g., a circularizable probe or probe set or a first and second linear probe) contains one or more barcodes. In some embodiments, the first nucleic acid sequence is in a first nucleic acid strand comprising a barcode sequence, and/or wherein the second nucleic acid sequence is in a second nucleic acid strand comprising a barcode sequence. In some embodiments, the nucleic acid molecule, probe, or first and second probe comprises a barcode sequence. In some embodiments, the first nucleic acid sequence and/or the second nucleic acid sequence comprises a barcode sequence or portion thereof. In some embodiments, one or more barcodes are indicative of a sequence in the target nucleic acid (e.g., a sequence of a region of interest such as a single nucleotide of interest (e.g., SNPs or point mutations), a dinucleotide sequence, or a short sequence of about 5 nucleotides in length). In some embodiments, the probe or probe set comprises 1, 2, 3, 4, 5, 6, 7, 8, 9, 10 or more barcode sequences. The barcode sequences may be positioned anywhere within the polynucleotides. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same polynucleotide are separated from one another by at least a phosphodiester bond (e.g., they may be immediately adjacent to each other but do not overlap), such as 1, 2, 3, 4, 5, 6, 7, 8, 9, 10 or more nucleotides apart.

Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during in situ detection of the sample.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the embodiments herein, a region (e.g., hybridization regions) associated with the target nucleic acid can comprise one or more barcode sequences or complements thereof. In any of the embodiments herein, the sequence of the rolling circle amplification product can comprise one or more barcode sequences or complements thereof. In any of the embodiments herein, a ligated probe or ligated oligonucleotide can comprise one or more barcode sequences or complements thereof. In any of the embodiments herein, the one or more barcode sequences can comprise a barcode sequence corresponding to the target nucleic acid. In any of the embodiments herein, the one or more barcode sequences can comprise a barcode sequence corresponding to the sequence of interest, such as variant(s) of a single nucleotide of interest.

The barcode sequences may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of target nucleic acids or products thereof is less than the number of distinct targets (e.g., nucleic acid analytes and/or protein analytes) of the target nucleic acids or products thereof, and yet the distinct targets may still be uniquely identified from one another, e.g., by encoding a probe with a different combination of barcode sequences. However, not all possible combinations of a given set of barcode sequences need be used. For instance, each target nucleic acid or product thereof may contain 1, 2, 3, 4, 5, 6, 7, 8, 9, 10, 11, 12, 13, 14, 15, etc. or more barcode sequences. In some embodiments, a population of target nucleic acid probes or products thereof may each contain the same number of barcode sequences, although in other cases, there may be different numbers of barcode sequences present on the various target nucleic acids. In some embodiments, the barcode sequences or any subset thereof in the population of target nucleic acid probes or products thereof can be independently and/or combinatorially detected and/or decoded.

As an illustrative example, a first target nucleic acid product (e.g., a ligated circular probe) may contain a first set of barcode sequences (e.g., first and second barcode sequences) and a second target nucleic acid product (e.g., a ligated circular probe) may contain a second set of barcode sequences (e.g., third and fourth barcode sequences). The first and second target nucleic acid products comprises barcode sequences that are different from each other. Such target nucleic acid products may thereby be distinguished by determining the various barcode sequence combinations present or associated with a target nucleic acid product at a given location in a sample.

In any of the preceding embodiments, barcodes can be analyzed (e.g., detected) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of detectably labeled probes (e.g., detectably labeled oligonucleotides labeled with a detectable moiety).

In some embodiments, the methods provided herein comprise contacting a biological sample with one or more probes and a single strand annealing protein. In some embodiments, the probe(s) each comprise a recognition sequence that hybridizes to a target sequence in a target nucleic acid. In some embodiments, the recognition sequence is at least about 6, at least about 8, at least about 10, at least about 15, at least about 20, at least about 25, at least about 30, at least about 35, or at least about 40 nucleotides in length. In some embodiments, the recognition sequence is between about 4 and 50, between about 4 and about 40, between about 4 and about 30, between about 8 and about 50, between about 8 and about 40, between about 10 and about 30, or between about 10 and about 25 nucleotides in length. In some embodiments, the probe(s) are contacted with the biological sample simultaneously or sequentially with a single strand annealing protein. In some embodiments, the probe(s) are contacted with the biological sample as complexes with the single strand annealing protein. In some embodiments, the probes are detectably labeled probes as shown in FIG. 3. In some embodiments, the probes comprise a binding site for hybridization of one or more intermediate probes and/or hybridization of one or more detectably labeled oligonucleotides. In some embodiments, a detectably labeled oligonucleotide comprises a hybridization sequence corresponding to a signal associated with the detectably labeled oligonucleotide, and a detectable moiety for producing the signal associated with the detectably labeled oligonucleotide. In some embodiments, the detectably labeled oligonucleotide(s) and/or intermediate probe(s) are contacted with the biological sample in the presence of a single strand annealing protein. In some embodiments, the detectably labeled oligonucleotide(s) and/or intermediate probe(s) is/are bound to the single strand annealing protein.

In some embodiments, the detectable moiety is modifiable. A detectable moiety or label associated with any of the probes described herein can be a fluorophore, a radioisotope, a molecule capable of a colorimetric reaction, or any other suitable molecule or compound capable of detection. In some embodiments, the detectable label is a fluorescent moiety. In some embodiments, the detectable moiety is a chemical moiety for direct chemical, optical, or enzymatic detection. In some embodiments, the detectable moiety is a chemical moiety for direct chemical, optical, or enzymatic modification. In some embodiments, the detectable moiety is a chemical moiety for downstream chemical, optical, or enzymatic detection. In some embodiments, the detectable moiety is detectable by an optical (e.g., a dye or light) or a non-optical method. In some embodiments, a chemical moiety for chemical detection is biotin, DIG, or various other molecules that can be detected with a secondary binding molecule. In some embodiments, a chemical moiety for chemical detection is a di-thio linker. In some embodiments, a chemical moiety for optical detection is a fluorophore. In some embodiments, chemical moiety for optical modification is biotin, DIG, or various other molecules that can be detected with a secondary binding molecule coupled to an enzyme (e.g., HRP) for signal amplification. In some embodiments, a chemical moiety for enzymatic modification is modified nucleotide such as dUTP, which is cleavable using a combination of uracil-DNA glycosylase and AP endonuclease catalyzing enzymes. In some embodiments, the detectable moiety is joined or associated, directly or indirectly via a linker, to the probe.

In some aspects, the provided methods involve analyzing, e.g., detecting or determining, one or more sequences present in the polynucleotides and/or in an amplification product, such as in an amplification product of a circularized probe, which may comprise one or more barcode sequences. In some embodiments, the analysis comprises determining the sequence of all or a portion of the ligation and/or amplification product. In some embodiments, the analysis comprises detecting a sequence present in the amplification product. In some embodiments, the sequence of all or a portion of the amplification product is indicative of the identity of a region (e.g., a single nucleotide) of interest in a target nucleic acid. In some embodiments, the analysis can be used to correlate a sequence detected in an amplification product to a circularizable probe (e.g., via a barcode). In some embodiments, the analysis can be used to correlate a sequence detected in a ligation product to a probe set of a first and second probe (e.g., via a barcode). In some embodiments, the detection of a sequence can provide information regarding the location and/or identity of an associated target nucleic acid (e.g., hybridized by the probe(s)) in a sample. In some embodiments, due to amplification of one or more polynucleotides (e.g., a circularizable probe), particular sequences present in the amplification product or complementary sequences thereof can be detected even when a polynucleotide is present at low levels before the amplification. For example, the number of copies of the barcode sequence(s) and/or a complementary sequence thereof is increased by virtue of the amplification of a probe comprising the barcode sequence(s) and/or complementary sequence thereof, thereby enabling specific and sensitive detection of a signal indicative of the identity of a short region (e.g., a single nucleotide) of interest in a target nucleic acid. In particular embodiments, the amplification product is an in situ rolling circle amplification (RCA) product of a circularized probe.

In some embodiments, the first and second nucleic acid sequences is comprised in a library of polynucleotides comprising different first nucleic acid sequences and/or different second nucleic acid sequences (e.g., a library of circularizable probes comprising different first nucleic acid sequences and/or second nucleic acid sequences, or a library of first and second probes comprising different first nucleic acid sequences and/or different second nucleic acid sequences). In some embodiments, the library of polynucleotides comprises a first circularizable probe comprising sequences capable of hybridizing to a first target nucleic acid within the sample. In some embodiments, the library of polynucleotides further comprises a second circularizable probe comprising sequences capable of hybridizing to a second target nucleic acid within the sample. In some aspects, the library of polynucleotides are contacted with a plurality of single strand annealing proteins to promote binding to the target nucleic acids. In some aspects, the library of polynucleotides undergo ligation, amplification, and detection steps. In some aspects, the library of ligated and amplified polynucleotides or products are not detectable or detectably labeled. The library of ligated polynucleotides or products thereof may be contacted with a plurality of detectably labeled probes comprising detectable moieties (e.g., first and second detectable moieties such as first and second fluorescence moieties), or a plurality of probes comprising binding sites for one or more intermediate probes and/or detectably labeled oligonucleotides, wherein the detectably labeled oligonucleotides comprise detectable moieties. The signals emitted by the library of polynucleotides or products thereof are detected in situ at one or more locations in the sample. In some embodiments, the methods provided herein are used for multiplex in situ hybridization or multiplex in situ sequence detection.

In some aspects, the provided methods involve a step of contacting, or hybridizing, one or more polynucleotides, such as any one of the probes described herein, to a cell or a sample containing a target nucleic acid with a region (e.g., single nucleotide) of interest in order to form a ligatable hybridization complex. In some aspects, the provided methods comprise contacting the polynucleotides with single strand annealing proteins. In some aspects, the provided methods comprise one or more steps of ligating the ends of polynucleotides, for instance of ligating the ends of a circularizable probe to form a circularized probe. In some aspects, the one or more steps of ligating the ends of polynucleotides, comprise ligating the ligatable ends of a probe set to form a linear probe. In some aspects, the provided methods involve a step of amplifying one of the polynucleotides (e.g., a padlock probe, a circularized probe or a linear probe produced therefrom), to generate an amplification product. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve performing one or more of the steps described herein, simultaneously and/or sequentially.

In some embodiments, a circular construct (e.g., ligated oligonucleotide or a ligated probe) is formed using ligation. In some embodiments, the circular construct is formed using template primer extension followed by ligation. In some embodiments, the ligated probe is generated using the target nucleic acid as template. In some embodiments, the circular construct is formed by providing an insert between ends to be ligated. In some embodiments, the circular construct is formed using a combination of any one of the foregoing. In some embodiments, the ligation is a DNA-DNA templated ligation. In some embodiments, the ligation is an RNA-RNA templated ligation. In some embodiments, the ligation is a RNA-DNA templated ligation. In some embodiments, a splint is provided as a template for ligation. In some embodiments, the circular construct is directly hybridized to the target nucleic acid. In some embodiments, the circular construct is formed from a modified circularizable probe or probe set (e.g., any of the circularizable probes or probe sets described herein). In some embodiments, the circular construct is formed from a probe or probe set capable of DNA-templated ligation. See, e.g., U.S. Pat. No. 8,551,710, the content of which is herein incorporated by reference in its entirety.

In some embodiments, the method comprises using a circular or circularizable construct hybridized to the target nucleic acid comprising the region of interest to generate a product (e.g., comprising a sequence of the region of interest associated with the target nucleic acid). In some aspects, the product is generated using RCA. In any one of the embodiments herein, the method can comprise ligating the ends of a circularizable probe hybridized to the target RNA to form a circularized probe. In any one of the embodiments herein, the method can further comprise generating a rolling circle amplification product of the circularized probe. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In any one of the embodiments herein, the method can further comprise detecting a signal associated with the rolling circle amplification product in the biological sample. In some embodiments, a ligation product of a first, second probe, and third probe is generated. In some aspects, the ligation product or a derivative thereof (e.g., extension product) can be detected. In some cases, RCA is not performed.

In some embodiments, the target nucleic acid is at a location in a biological sample and the ligated probe is generated and optionally amplified at the location in the biological sample. Following formation of, e.g., the circularized probe, in some instances, an amplification primer is added. In other instances, the amplification primer is added with the circularizable probes. In some instances, the amplification primer may also be complementary to the target nucleic acid and the circularizable probe. In some embodiments, a washing step is performed to remove any unbound probes, primers, etc. In some embodiments, the wash is a stringency wash. Washing steps can be performed at any point during the process to remove non-specifically bound probes, probes that have ligated, etc. In some embodiments, the stringency is increased during the hybridization of the probe or probe set to the target nucleic acid, reducing or negating the need of performing a stringency wash.

Upon addition of a DNA polymerase in the presence of appropriate dNTP precursors and other cofactors, the amplification primer is elongated by replication of multiple copies of the template (e.g., a concatemer of the template is generated). This amplification product can be detected using, e.g., the secondary and higher order probes and detectably labeled oligonucleotides described herein. In some embodiments, the sequence of the amplicon or a portion thereof, is determined or otherwise analyzed, for example by using detectably labeled probes and imaging. The detection or analysis of the amplification products can comprise fluorescent in situ hybridization.

In any one of the embodiments herein, the method can further comprise generating the product of the circularized probe in situ in the biological sample. In any one of the embodiments herein, the product can be generated using rolling circle amplification (RCA). In any one of the embodiments herein, the RCA can comprise a linear RCA, a branched RCA, a dendritic RCA, or any combination thereof. In any one of the embodiments herein, the product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include but are not limited to linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833, US 2018/0051322, US 2019/0024144, US 2021/0324450, US 2022/0228196, and US 2017/0219465, each of which is incorporated herein by reference in its entirety for all purposes. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

The present disclosure in some aspects provides a method for nucleic acid ligation, comprising: a) providing a first nucleic acid strand, a second nucleic acid strand, and a third nucleic acid strand; wherein the first nucleic acid strand comprises a first sequence that hybridizes to a first hybridization region of the third nucleic acid strand, and the second nucleic acid strand comprises a second sequence that hybridizes to a second hybridization region of the third nucleic acid strand wherein the first nucleic acid strand, second nucleic acid strand and/or the third nucleic acid strand are/is bound to a single strand annealing protein; and b) ligating the first nucleic acid strand and second nucleic acid strand using the third nucleic acid strand as template to generate a ligated oligonucleotide comprising the first sequence and the second sequence.

The present disclosure in some aspects provides a method for analyzing a biological sample, comprising: a) providing a plurality of first nucleic acid strands, and a plurality of second nucleic acid strands; wherein the plurality of first nucleic acid strands comprise a plurality of first sequences that hybridize to a plurality of first hybridization regions, and the plurality of second nucleic acid strands comprise a plurality of second sequences that hybridize to a plurality of second hybridization regions, wherein the first and second hybridization regions are within a plurality of target nucleic acids in the biological sample, wherein the plurality of first nucleic acid strands and/or second nucleic acid strands are bound to a plurality of single strand annealing proteins; and b) ligating the plurality of first nucleic acid strands and the plurality of second nucleic acid strands using plurality of target nucleic acids as templates to generate a plurality of ligated oligonucleotides comprising the plurality of first sequences and the plurality of second sequences. In some embodiments, the plurality of first and/or second sequences comprise a plurality of barcode sequences. In some embodiments, the method comprises detecting the plurality of ligated probe or product thereof, wherein the plurality of ligated oligonucleotides are detected in multiple cycles at different locations in the biological sample. In some embodiments, a probe set comprising a linear first and a second probe can be ligated together to form a linear ligated probe product (e.g., ligated oligonucleotide) and the linear ligated probe product is captured on an array for analysis.

In some embodiments, provided herein is a method for nucleic acid ligation and or nucleic acid processing, comprising: a) contacting a biological sample with a probe or a probe set comprising a first sequence, wherein the probe hybridizes to a first hybridization region of target nucleic acid; b) ligating the first sequence of the probe or probe set to a second sequence hybridized to a second hybridization region of the target nucleic acid in the presence of a single strand annealing protein bound to the first sequence and/or the second sequence, wherein the second hybridization region is adjacent to the first hybridization region in the target nucleic acid, wherein the second sequence is a part of the probe or probe; and c) detecting the ligated probe or product thereof comprising the first sequence and second sequence or a product thereof.

In some embodiments, provided herein is a method for analyzing a target RNA, comprising: a) contacting the target RNA with a circularizable DNA probe set comprising a first sequence, wherein the probe hybridizes to a first hybridization region of target nucleic acid; b) ligating the first sequence of the circularizable DNA probe set to a second sequence hybridized to a second hybridization region of the target RNA in the presence of a single strand annealing protein bound to the first sequence and/or the second sequence, wherein the second sequence is a part of the probe or probe, wherein the second hybridization region is adjacent to the first hybridization region in the target RNA; and c) detecting the ligated circularizable DNA probe or product thereof comprising the first sequence and second sequence or a product thereof.

The present disclosure in some aspects provides an exemplary method for analyzing a biological sample, comprising: contacting a DNA concatemer (e.g., a rolling circle amplification product) with a probe, wherein the probe is bound to a single strand annealing protein, and detecting a signal associated with the probe, thereby detecting the DNA concatemer in the biological sample. In some embodiments, the signal is associated with the probe via direct or indirect binding of a detectably labeled oligonucleotide to a binding site in the probe (e.g., in an overhang region of the probe). In some embodiments, prior to the contacting step, the biological sample comprises a target nucleic acid hybridized to a circularized probe. In some embodiments, the circularized probe is amplified to generate the DNA concatemer. In some embodiments, the target nucleic acid is RNA and the circularizable probe comprises DNA. In some embodiments, the method comprises hybridizing the circularizable probe to the target nucleic acid in the presence of a single strand annealing protein.

### b) Detection of Ligation Products

In any of the embodiments herein, the detecting step can comprise contacting the biological sample with a one or more detectably labeled probes that directly or indirectly bind to the hybridized and/or ligated probe or probe set, or a rolling circle amplification product thereof. In some embodiments, the step comprises detecting a first signal or absence thereof associated with detectably labeled probe (e.g., detectably labeled oligonucleotide) at a location in the biological sample. A first signal may be detected using any suitable imaging technique described herein.

In some embodiments, the biological sample is further contacted with a secondary probe that hybridizes to the ligated probe or a product thereof. In some embodiments, the secondary probe is a detectably labeled probe (e.g., a detectably labeled oligonucleotide) comprising a detectable moiety. In some embodiments, the detectable moiety is a fluorophore. In some embodiments, the method comprises contacting the sample with detectably labeled probes (e.g., detectably labeled oligonucleotides). In some aspects, the detectably labeled probes are bound to single strand annealing proteins (as shown in FIG. 3). The detectably labeled probes bound to the single strand annealing proteins promote the annealing of the detectably labeled probes to the ligated probes or products thereof described herein. The ligation efficiency of the single strand annealing protein bound detectably labeled probes to the ligated probes (e.g., ligated oligonucleotides) is increased as compared to the ligation efficiency of the detectably labeled probes without being bound to the single strand annealing proteins.

In some embodiments, the secondary probe comprises an overhang region that does not hybridize to the ligated probe or product thereof. In some embodiments, the method comprises contacting the biological sample with a detectably labeled oligonucleotide, wherein the detectably labeled oligonucleotide comprises a detectable moiety and a sequence that hybridizes to the overhang region of the secondary probe. In some embodiments, the detectable moiety is a fluorophore. In some embodiments, the secondary probe is bound to a single strand annealing protein, and the single strand annealing protein promotes hybridization of the secondary probe to the ligated probe or product thereof. In some embodiments, the detectably labeled oligonucleotide is bound to a single strand annealing protein, and the single strand annealing protein promotes hybridization of the detectably labeled oligonucleotide to the secondary probe.

In some embodiments, the detecting comprises: contacting the biological sample with one or more detectably labeled probes that directly or indirectly bind to one or more barcode sequences or complements thereof in a plurality of ligated oligonucleotides or plurality of products thereof, detecting signals associated with the one or more detectably labeled probes, and removing the one or more detectably labeled probes.

In some embodiments, the detecting comprises: contacting the biological sample with one or more intermediate probes that directly or indirectly bind to one or more barcode sequences or complements thereof in the plurality of ligated oligonucleotides or plurality of products thereof, wherein the one or more intermediate probes are detectable using one or more detectably labeled probes, and detecting signals associated with the one or more detectably labeled probes. In some embodiments, the method further comprising removing the one or more intermediate probes and/or the one or more detectably labeled probes.

In some embodiments, a detectably labeled probe (e.g., detectably labeled first and/or second probe) is labeled with a detectable moiety. In some embodiments, a detectably labeled probe comprises one detectable moiety. In some embodiments, a detectably labeled probe comprises two or more detectable moieties. In some embodiments, a detectably labeled probe has one detectable moiety. In some embodiments, a detectably labeled probe has two or more detectable moiety.

In some embodiments, the detection may be spatial, e.g., in two or three dimensions. In some embodiments, the detection may be quantitative, e.g., the amount or concentration of a target nucleic acid may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc.

In some embodiments, each detectably labeled probe (e.g., detectably labeled first and/or second probe) in a set has a different target, e.g., a transcript or DNA locus. In some embodiments, all detectably labeled probes (e.g., detectably labeled first and/or second probes) for a target in a set have the same detectable moieties. In some embodiments, all detectably labeled probes are labeled in the same way. In some embodiments, all the detectably labeled probes for a target have the same fluorophore.

In some aspects, the methods disclosed herein involve the use of one or more probes (e.g., first and/or second probes comprising the first and second nucleic acid sequences, respectively) that directly or indirectly hybridize to a target nucleic acid, such as an RNA molecule or a derivative thereof. In some aspects, a sample is contacted with a plurality of barcoded probe sets, each for analyzing an analyte (e.g., biomarker) panel. The sample can be contacted, in separate rounds, with different detectably labeled probes (e.g., detectably labeled first and/or second probes) for analyzing one or more sets in the barcoded probe sets (and thereby analyzing the one or more analyte panels corresponding to the one or more sets). The detectably labeled probes may be changed between rounds while the barcoded probes remain bound to analytes in the sample.

In some aspects, the provided methods comprise imaging the biological sample comprising the hybridized and/or ligated probe or probe set, or an amplification product (e.g., RCP), for example, via binding of the detectably labeled probe and detecting the detectable label. In some embodiments, the detectably labeled probe comprises a detectable label that can be measured and quantitated. The terms “label” and “detectable label” comprise a directly or indirectly detectable moiety that is associated with (e.g., conjugated to) a molecule to be detected, e.g., a detectably labeled probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectably labeled probe containing a detectable label can be used to detect one or more target nucleic acids and/or amplification products (e.g., RCP) described herein. In some embodiments, the methods involve incubating the detectably labeled probe containing the detectable label with the sample and detecting the label, e.g., by imaging.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and US In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Any suitable methods for custom synthesis of nucleotides having other fluorophores (See, Henegariu et al. (2000) Nature Biotechnol. 18:345) may be used.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleic acid probe (e.g., detectable labeled first and/or second probe), and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NETS) derivatized fluorescent dye. In general, any member of a conjugate pair may be incorporated into a detection polynucleotide provided that a detectably labeled conjugate partner can be bound to permit detection. In some embodiments, the term antibody comprises an antibody molecule of any class, or any sub-fragment thereof, such as a Fab.

Other suitable labels for a nucleic acid probe (e.g., detectable labeled first and/or second probe) may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In any of the embodiments herein, the method can further comprise imaging the biological sample to detect a probe (e.g., circularizable probe or first and/or second probe) hybridized to a target nucleic acid or product thereof. In any of the embodiments herein, a sequence of the ligation product, rolling circle amplification product, or other generated product can be analyzed in situ in the biological sample. In any of the embodiments herein, the imaging can comprise detecting a signal associated with a fluorescently labeled probe (e.g., detectably labeled first and/or second probes) that directly or indirectly binds to a target nucleic acid or product thereof (e.g., rolling circle amplification product of the circularized probe). In any of the embodiments herein, the sequence of the ligation product, rolling circle amplification product, or other generated product can be analyzed by sequential hybridization and in situ detection.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectably labeled probe (e.g., detectably labeled first and/or second probe). In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the barcodes of the target nucleic acids or products thereof are analyzed using one or more decoding schemes to decode the signals, such as fluorescence, for sequence determination. In any of the embodiments herein, barcodes can be analyzed (e.g., detected) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labeled probes (e.g., first, second, third, fourth, . . . , nth probes). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):e112; U.S. Pat. No. 10,457,980 B2; US 2016/0369329 A1; US 2021/0017587; and US 2017/0220733 A1, all of which are herein incorporated by reference in their entireties. In some embodiments, these assays enable signal amplification, combinatorial decoding, and error correction schemes at the same time.

In some aspects, the analysis and/or sequence determination can be carried out at room temperature for best preservation of tissue morphology with low background noise and error reduction. In some embodiments, the analysis and/or sequence determination comprises eliminating error accumulation as in situ detection proceeds.

In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data

In some embodiments, an exemplary workflow for analyzing a biological sample comprises a) providing a first nucleic acid strand, a second nucleic acid strand, and a third nucleic acid strand; wherein the first nucleic acid strand comprises a first sequence that hybridizes to a first hybridization region of the third nucleic acid strand, and the second nucleic acid strand comprises a second sequence that hybridizes to a second hybridization region of the third nucleic acid strand wherein the first nucleic acid strand, second nucleic acid strand and/or the third nucleic acid strand are/is bound to a single strand annealing protein; and b) ligating the first nucleic acid strand and second nucleic acid strand using the third nucleic acid strand as template to generate a ligated oligonucleotide comprising the first sequence and the second sequence. In some embodiments, the single strand annealing protein promotes ligation and/or binding of the first sequence and/or the second sequence. In some embodiments, the method comprises detecting the ligated probe or product thereof, wherein the detecting comprises determining a sequence of the probe, or a complementary sequence or a product (e.g., a hybridization product, a ligation product, an extension product (e.g., by a DNA or RNA polymerase), a replication product, a transcription/reverse transcription product, and/or an amplification product) of the probe. In some embodiments, the target nucleic acid is at a location in a biological sample and the ligated probe is generated and optionally amplified at the location in the biological sample, and wherein the ligated probe and/or the product thereof is detected at the location in the biological sample, optionally wherein the product is a rolling circle amplification (RCA) product.

In some embodiments, an exemplary workflow for analyzing a biological sample comprises a) contacting a biological sample with a probe or a probe set comprising a first sequence, wherein the probe hybridizes to a first hybridization region of target nucleic acid; b) ligating the first sequence of the probe or probe set to a second sequence hybridized to a second hybridization region of the target nucleic acid in the presence of a single strand annealing protein bound to the first sequence and/or the second sequence, wherein the second hybridization region is adjacent to the first hybridization region in the target nucleic acid, wherein the second sequence is a part of the probe or probe; and c) detecting the ligated probe or product thereof comprising the first sequence and second sequence or a product thereof. In some embodiments, the single strand annealing protein promotes ligation and/or binding of the first sequence and/or the second sequence. In some embodiments, the method comprises detecting the ligated probe or product thereof, wherein the detecting comprises determining a sequence of the probe, or a complementary sequence or a product (e.g., a hybridization product, a ligation product, an extension product (e.g., by a DNA or RNA polymerase), a replication product, a transcription/reverse transcription product, and/or an amplification product) of the probe. In some embodiments, the target nucleic acid is at a location in a biological sample and the ligated probe is generated and optionally amplified at the location in the biological sample, and wherein the ligated probe and/or the product thereof is detected at the location in the biological sample, optionally wherein the product is a rolling circle amplification (RCA) product.

In some embodiments, a linear ligated probe product (e.g., ligated first probe to the second probe is captured on an array and analyzed (e.g., by sequencing). In some embodiments, after generation of the linear ligated probe product from the probe or probe sets described herein, the assay may further comprise one or more optional steps for transferring the probes (or a product or derivative thereof) to an array. In some embodiments, the probes (e.g., first probe and second probe) can be ligated in the presence of a single strand annealing protein and captured on an array. In some embodiments, a product (e.g., extension product) or derivative of the ligated probes can be transferred to an array.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array. Non-limiting aspects of spatial analysis methodologies and compositions are described in U.S. Pat. Nos. 10,774,374, 10,724,078, 10,480,022, 10,059,990, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, 2022/0010367, 2023/0143569, 2023/0159995, WO 2018/091676, WO 2020/176788, Rodrigues et al., Science 363(6434):1463-1467, 2019; Lee et al., Nat. Protoc. 10(3):442-458, 2015; Trejo et al., PloS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

In some cases, spatial analysis can be performed by attaching and/or introducing a molecule having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. Some such methods of spatial analysis are described in Section (III) of WO 2020/176788 and/or U.S. Patent Application Publication Nos. 2020/0277663, 2022/0010367, 2023/0143569, and 2023/0159995, each of which is incorporated herein by reference in its entirety for call purposes.

In some cases, spatial analysis can be performed by detecting multiple oligonucleotides (e.g., probe or probe set as described herein) that hybridize to a target nucleic acid in the presence of a single strand annealing protein. In some instances, for example, spatial analysis can be performed by hybridization of two oligonucleotides (e.g., a first probe and a second probe comprising the first and second nucleic acid sequences, respectively) to adjacent sequences on an analyte (e.g., an RNA molecule, such as an mRNA molecule). In some instances, the oligonucleotides are DNA molecules. After hybridization of the probe(s) in the presence of a single strand annealing protein to the analyte, a ligation product is generated. In some instances, the two oligonucleotides (e.g., probes) hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product (or a derivative thereof) can then be captured by an oligonucleotide comprising a capture sequence complementary to a sequence of the ligated probe(s) on an array, optionally amplified, and sequenced, thus determining the location and optionally the abundance of the analyte in the biological sample. In some embodiments, the ligated probe(s) comprise a complementary capture sequence. In some instances, a ligated probe comprises an overhang region (e.g., a region that does not hybridize to the target nucleic acid) comprising the complementary capture sequence. In some embodiments, the capture sequence comprises a polyA sequence.

In some embodiments, after ligating, the method can comprise a step of removing unligated molecules of the probe or probe set (e.g., first and/or second probes that are not ligated). In some embodiments, ligation of the probe or probe set (e.g., the first and second probe) stabilizes hybridization of the probe(s) to the target nucleic acid. In some embodiments, the method can comprise one or more stringency washes to remove unligated probes (optionally, prior to migrating probes to capture oligonucleotides).

In some instances, the ligated probe product can be migrated toward a oligonucleotide comprising a capture sequence (e.g., a capture probe immobilized, directly or indirectly, on a substrate) on an array, optionally amplified, and sequenced to determine the spatial location of a molecule of interest (for example, an mRNA molecule). In some instances, the migration toward the capture probe can take place prior to and/or during probe ligation (e.g., ligation of a probe or probe set). After hybridization and ligation, the ligated probe product can be extended to make a copy of the additional components (for example, the spatial barcode) of the capture probe.

A capture probe can be any molecule capable of capturing (directly or indirectly) a probe or product corresponding to an analyte in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication Nos. 2020/0277663, 2022/0010367, 2023/0143569, and 2023/0159995. Generation of capture probes can be achieved by any appropriate method, including those described in Section (II)(d)(ii) of WO 2020/176788 and/or U.S. Patent Application Publication Nos. 2020/0277663, 2022/0010367, 2023/0143569, and 2023/0159995, each of which is incorporated herein by reference in its entirety for all purposes.

In some embodiments, at least one of the probes for binding a target nucleic acid (e.g., probe or probe set) comprise a sequence complementary to a sequence comprised by a oligonucleotide comprising a capture sequence (e.g. capture probe). In some embodiments, a plurality of the probes (e.g., first or second probes, or ligated probes generated from the first and second probes) comprise a common sequence for hybridizing to a capture sequence on the oligonucleotide (e.g., capture probe). In some embodiments, the capture probes are spatially-barcoded capture probes attached to a substrate (e.g., array). In some instances, the spatially-barcoded capture probes described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence). In some examples, a ligation product (or a derivative thereof) can be captured by the oligonucleotides comprising the capture sequence (e.g. capture probes) and associated with a spatial barcode, optionally amplified, and sequenced, thus determining the location of the target nucleic acid. In some cases, the spatially-barcoded capture probes can be attached to functional sequences described herein (such as sequence specific flow cell attachment sequences) prior to analysis. In some cases, the spatially barcoded analyte (or a product or derivative thereof) can be released from the array prior to analysis. In some cases, the spatially barcoded analyte can be further processed and subjected to one or more reactions prior to analysis (e.g., extension, amplification, or sequencing).

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication Nos. 2020/0277663, 2022/0010367, 2023/0143569, and 2023/0159995 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

In some embodiments, an extended capture probe is a capture probe having additional nucleotides added to the terminus (e.g., 3′ or 5′ end) of the capture probe thereby extending the overall length of the capture probe. For example, an extended 3′ end can comprise additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication Nos. 2020/0277663, 2022/0010367, 2023/0143569, and 2023/0159995. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication Nos. 2020/0277663, 2022/0010367, 2023/0143569, and 2023/0159995. Some quality control measures are described in Section (II)(h) of WO 2020/176788 and/or U.S. Patent Application Publication Nos. 2020/0277663, 2022/0010367, 2023/0143569, and 2023/0159995.

Typically, for spatial array-based methods, a substrate functions as a support for direct or indirect attachment of capture probes to features of the array. A feature can be an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication Nos. 2020/0277663, 2022/0010367, 2023/0143569, and 2023/0159995. Exemplary features and geometric attributes of an array can be found in Sections (II)(d)(i), (II)(d)(iii), and (II)(d)(iv) of WO 2020/176788 and/or U.S. Patent Application Publication Nos. 2020/0277663, 2022/0010367, 2023/0143569, and 2023/0159995.

Generally, analytes and/or corresponding probes can be captured when contacting a biological sample with a substrate including capture probes (e.g., a substrate with capture probes embedded, spotted, printed, fabricated on the substrate, or a substrate with features (e.g., wells) comprising capture probes). In the context of spatial array capture, contacting a biological sample with a substrate refers can comprise any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication Nos. 2020/0277663, 2022/0010367, 2023/0143569, and 2023/0159995.

During analysis of spatial information, sequence information for a spatial barcode associated with an analyte (e.g., ligated first and second probe or product or derivative thereof) is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication Nos. 2020/0277663, 2022/0010367, 2023/0143569, and 2023/0159995. See, for example, the Exemplary embodiment starting with “In some non-limiting examples of the workflows described herein, the sample can be immersed . . . ” of WO 2020/176788 and/or U.S. Patent Application Publication Nos. 2020/0277663, 2022/0010367, 2023/0143569, and 2023/0159995. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication Nos. 2020/0277663, 2022/0010367, 2023/0143569, and 2023/0159995, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320 and/or U.S. Patent Application Publication No. 20220049294, each of which is incorporated herein by reference in its entirety for all purposes.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064, U.S. Patent Application Publication Nos. 2019/0392503 and 2021/0150707, each of which is incorporated herein by reference in its entirety for all purposes.

Prior to transferring analytes (or corresponding probes or products generated therefrom) from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in US 2021/0155982 A1, each of which is incorporated herein by reference in its entirety for all purposes.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, U.S. Patent Application Publication Nos. 2022/0049294, 2020/0090801, and/or U.S. patent application Ser. No. 16/951,843, each of which is incorporated herein by reference in its entirety for all purposes. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

In any one of the embodiments herein, a ligated linear probe (e.g., generated from a first and second probe described herein) can comprise one or more barcode sequences or complements thereof. In some embodiments, a ligated linear probe can comprise an overhang region (e.g., a region that does not hybridize to the target nucleic acid) comprising one or more barcode sequences or complements thereof, which can be detected according to any of the methods described herein. In some embodiments, a ligated linear probe can be released from the target nucleic acid (e.g., by RNase H digestion) prior to detecting a sequence of the ligated linear probe. In any one of the embodiments herein, the one or more barcode sequences can comprise a barcode sequence corresponding to the target nucleic acid. In any one of the embodiments herein, the one or more barcode sequences can comprise a barcode sequence corresponding to the sequence of interest, such as variant(s) of a single nucleotide of interest.

In some aspects, any of the probe(s) described herein can comprise one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or UMI). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some cases, sequencing can be performed after the analytes are released from the biological sample. In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232, each of which is incorporated herein by reference in its entirety for all purposes.

In some embodiments, sequence analysis of nucleic acids (e.g., nucleic acids such as probes or RCA products comprising barcode sequences) can be performed by sequential hybridization (e.g., sequencing by hybridization and/or sequential in situ fluorescence hybridization). Sequential fluorescence hybridization can involve sequential hybridization of detectably labeled probes comprising an oligonucleotide and a detectable label. In some embodiments, a method disclosed herein comprises sequential hybridization of the detectably labeled probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectably labeled probes are described in US 2019/0161796, US 2020/0224244, US 2022/0010358, US 2021/0340618, US 2023/0039899, and WO 2021/138676, all of which are incorporated herein by reference in their entireties. In some embodiments, the methods provided herein can include analyzing a sequence of a ligated probe or product thereof by sequential hybridization and detection with a plurality of labeled probes (e.g., detectably labeled oligonucleotides).

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 6,969,488; 6,172,218; and 6,306,597.

In some embodiments, the barcodes of the probes (e.g., the circularizable probe or a spatially barcoded analyte comprising a sequence of the ligated probes) or complements or products thereof are targeted by detectably labeled oligonucleotides, such as fluorescently labeled oligonucleotides. In some embodiments, one or more decoding schemes are used to decode the signals, such as fluorescence, for sequence determination. In any one of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detectably labeled oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., Science; 348(6233):aaa6090 (2015); Gyllborg et al., Nucleic Acids Res (2020) 48(19):e112; U.S. Pat. No. 10,457,980 B2; US 2016/0369329 A1; US 2021/0017587; and US 2017/0220733 A1, all of which are herein incorporated by reference in their entireties. In some embodiments, these assays enable signal amplification, combinatorial decoding, and error correction schemes at the same time.

### c) Samples and Analytes

### I. Samples

A sample disclosed herein can be or be derived from any biological sample. Methods and compositions disclosed herein may be used for analyzing a biological sample, which may be obtained from a subject using any one of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, a section of a cell block or cell pellet, biopsy, a core biopsy, needle aspirate, or fine needle aspirate. The sample can be a single cell sample, such as a dissociated single cell sample. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 80, 90, or 100 μm or more. Typically, the thickness of a tissue section is between 1-100 μm, 1-μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any one of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe or probe set (e.g., a padlock probe), or a first and/or second nucleic acid strand. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., a padlock probe) or to generate a ligated probe or ligated oligonucleotide.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any one of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the content of which is herein incorporated by reference in its entirety.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, all of which are incorporated herein by reference in their entireties.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the contents of which are incorporated herein by reference in its entirety).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, all of which are incorporated herein by reference in their entireties.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the content of which is herein incorporated herein by reference in its entirety. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any one of a variety of methods (e.g., streptavidin beads).

Alternatively, one or more species of RNA can be down-selected (e.g., removed) using any one of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the content of which is incorporated herein by reference in its entirety). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire content of which is incorporated herein by reference in its entirety).

A biological sample may comprise one or a plurality of analytes of interest. Methods for performing multiplexed assays to analyze two or more different analytes in a single biological sample are provided.

### II. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a hybridization region. In some embodiments, the hybridization region may be endogenous to the sample, generated in the sample, added to the sample, or associated with an analyte in the sample. In some embodiments, the hybridization region is a single-stranded hybridization region (e.g., a sequence in a rolling circle amplification product). In some embodiments, the analytes comprise one or more hybridization regions. In one aspect, a first hybridization region is not identical to a second hybridization region. In another aspect, a first hybridization region is identical to one or more second hybridization regions. In some embodiments, the one or more hybridization region is comprised in the same analyte (e.g., nucleic acid) as the first hybridization region. Alternatively, the one or more second hybridization region is comprised in a different analyte (e.g., nucleic acid) from the first hybridization region.

### (ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a circularizable probe), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any one of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entireties.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any one of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, the content of which is herein incorporated by reference in its entirety. Likewise, protein and peptide biotinylation techniques have been developed and are readily available. See, e.g., U.S. Pat. No. 6,265,552, the content of which is herein incorporated by reference in its entirety. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### B. Spatial Array Generation

In some aspects, provided herein are methods for improving efficiency and specificity of nucleic acid ligation during spatial array generation. In some embodiments, the methods disclosed herein can be used to generate a spatial array. In some embodiments, a first nucleic acid strand is immobilized on a substrate (e.g., a planar substrate). In some embodiments, the substrate is a glass slide. In some embodiments, additional polynucleotides (e.g., second nucleic acid strands) comprising barcode sequences are ligated to the immobilized polynucleotides via splint sequences. In some embodiments, a single strand annealing protein (e.g., DrB or RecT) promotes ligation and/or binding of the polynucleotide sequences (e.g., first and second nucleic acid strands). In some aspects, ligated polynucleotides comprising spatial barcodes are generated on the substrate.

In some embodiments, a method provided herein further comprises a step of providing the substrate. A wide variety of different substrates can be used for the foregoing purposes. In general, a substrate can be any suitable support material. The substrate may comprise materials of one or more of the IUPAC Groups 4, 6, 11, 12, 13, 14, and 15 elements, plastic material, silicon dioxide, glass, fused silica, mica, ceramic, or metals deposited on the aforementioned substrates. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, quartz, metals, inorganic glasses, optical fiber bundles, and polymers, such as polystyrene, cyclic olefin copolymers (COCs), cyclic olefin polymers (COPs), polypropylene, polyethylene and polycarbonate. In some embodiments, the substrate is a glass substrate.

In some embodiments, a substrate comprising an array of first nucleic acid strands immobilized to the substrate is provided. The nucleic acid strands can be single-stranded. Nucleic acid strands on an array may be DNA or RNA. In some embodiments, the nucleic acid strands (e.g., the first nucleic acid sequence, the second nucleic acid sequence, and the third nucleic acid sequence in the strands) are DNA. In some embodiments, a plurality of first nucleic acid strands immobilized to the substrate comprise the same nucleotide sequence. In some embodiments, a plurality of first nucleic acid strands immobilized to the substrate comprises different nucleotide sequences.

In some embodiments, a first nucleic acid strand is immobilized on a substrate (e.g., a glass slide). In some embodiments, a second nucleic acid strand hybridized to a splint (e.g., a third nucleic acid strand) is provided as a partially double stranded duplex, as shown in FIG. 4. The first nucleic acid strand comprises a first sequence that hybridizes with the first hybridization region of the splint (e.g., third nucleic acid strand). The second nucleic acid strand comprises a second sequence that hybridizes to a second hybridization region of the splint (e.g., third nucleic acid strand). The method comprises ligating the first nucleic acid strand and second nucleic acid strand using the splint (e.g., third nucleic acid strand) as template. In some embodiments, the annealing of the first nucleic acid strand to the second nucleic acid strand is promoted by strand annealing proteins (e.g., DrB or RecT) bound to the first and second nucleic acid strands and/or the splint. The single strand annealing proteins promote ligation and/or binding of the first nucleic acid strand to the second nucleic acid strand. Ligation efficiency is increased by at least 10%, at least 20%, at least 30%, at least 40%, at least 50%, at least 60%, at least 70%, at least 80%, at least 90%, or by 100%. The first nucleic acid strand is ligated to the second nucleic acid strand by a ligase (such as a DNA ligase or a SplintR ligase). The ligase ligates the first nucleic acid strand to the adjacent second nucleic acid strands splinted by a complementary third nucleic acid strand. In some instances, the first nucleic acid strand and the second nucleic acid strand hybridize to sequences on the third nucleic acid strand that are not adjacent to one another. For example, hybridization of the first nucleic acid strand and the second nucleic acid strand creates a gap between the hybridized nucleic acid strands. In some aspects, the ligation is preceded by gap filling. The gap may be 1, 2, 3, 4, 5, or more nucleotides. In some instances, a polymerase (e.g., a DNA polymerase) can extend one of the nucleic acid strands prior to ligation. In some embodiments, the 3′ end and the 5′ ends of the first and second nucleic acid strands are ligated without gap filling. In some aspects, a ligated oligonucleotide comprising the first nucleic acid sequence and the second nucleic acid sequence is generated on the substrate (e.g., array). In some aspects, the ligated oligonucleotide comprising the first nucleic acid sequence and the second nucleic acid sequence is generated in a first cycle.

In some embodiments, the polynucleotides (e.g., first nucleic acid strand and/or second nucleic acid strand) can include various domains such as, spatial barcodes, unique molecular identifier (UMI)s, functional domains (e.g., sequencing handle), cleavage domains, and/or ligation handles. In some embodiments, the first nucleic acid strand further comprises a Read 1 (R1) primer sequence, a spatial barcode sequence or portion thereof and/or a UMI. In some embodiments, the second nucleic acid sequence comprises a barcode sequence or portion thereof (e.g., spatial barcode). In some embodiments, the second nucleic acid strand further comprises a splint sequence. The splint sequence hybridizes to a complementary sequence on the splint (e.g., a third nucleic acid strand disclosed herein).

A splint is an oligonucleotide that, when hybridized to other polynucleotides, acts as a “splint” to position the polynucleotides next to one another so that they can be ligated together. In some embodiments, the splint is DNA or RNA. The splint can include a nucleotide sequence that is partially complimentary to nucleotide sequences from two or more different oligonucleotides. In some embodiments, the splint assists in ligating a “donor” oligonucleotide and an “acceptor” oligonucleotide. In general, an RNA ligase, a DNA ligase, or another other variety of ligase is used to ligate two nucleotide sequences together.

Splints have been described, for example, in US20150005200A1. A splint may be used for ligating two oligonucleotides. The sequence of a splint may be configured to be in part complementary to at least a portion of the first oligonucleotides that are attached to the substrate and in part complementary to at least a portion of the second oligonucleotides. In one case, the splint can hybridize to the second oligonucleotide via its complementary sequence; once hybridized, the second oligonucleotide or oligonucleotide segment of the splint can then be attached to the first oligonucleotide attached to the substrate via any suitable attachment mechanism, such as, for example, a ligation reaction. The splint complementary to both the first and second oligonucleotides can then be then denatured (or removed) with further processing. The method of attaching the second oligonucleotides to the first oligonucleotides can then be optionally repeated to ligate a third, and/or a fourth, and/or more parts of the barcode onto the array with the aid of splint(s). In some embodiments, the splint is between 6 and 50 oligonucleotides in length, e.g., between 6 and 45, 6 and 40, 6 and 35, 6 and 30, 6 and 25, or 6 and 20 oligonucleotides in length. In some embodiments, the splint is between 15 and 50, 15 and 45, 15 and 40, 15 and 35, 15 and 30, 15 and 30, or 15 and 25 nucleotides in length.

A spatial barcode may comprise a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate. A spatial barcode can be part of a capture probe on an array generated herein. A spatial barcode can also be a tag attached to an analyte (e.g., a nucleic acid molecule) or a combination of a tag in addition to an endogenous characteristic of the analyte (e.g., size of the analyte or end sequence(s)). A spatial barcode can be unique. In some embodiments where the spatial barcode is unique, the spatial barcode functions both as a spatial barcode and as a unique molecular identifier (UMI), associated with one particular capture probe. Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode is added to, for example, a fragment of a DNA or RNA sample before sequencing of the sample. In some embodiments, a spatial barcode allows for identification and/or quantification of individual sequencing-reads. In some embodiments, a spatial barcode is a used as a fluorescent barcode for which fluorescently labeled oligonucleotide probes hybridize to the spatial barcode.

The polynucleotides (e.g., first nucleic acid strand and/or second nucleic acid strand) can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain). A UMI can be unique. A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 90% sequence identity (e.g., less than 80%, 70%, 60%, 50%, or less than 40% sequence identity) to the nucleic acid sequences across a substantial part (e.g., 80% or more) of the nucleic acid molecules in the biological sample. The UMI can include from about 6 to about 20 or more nucleotides within the sequence of capture probes, e.g., barcoded oligonucleotides in an array generated using a method disclosed herein. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter. These nucleotides can be contiguous, (e.g., in a single stretch of adjacent nucleotides), or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, the methods disclosed herein comprise additional ligation reactions with additional barcoded nucleic acid molecules (e.g., nucleic acid strands comprising a fourth, fifth, sixth, and/or seventh barcode sequence) in multiple cycles of annealing and ligation. In some embodiments, after ligating a first nucleic acid strand to the second nucleic acid strand and creating a ligation product (e.g., barcoded ligated oligonucleotide) in a first cycle, additional polynucleotide (e.g., fourth, fifth, and/or sixth nucleic acid strands) can be contacted to the array slide to extend the array in subsequent cycles. For instance, a fourth nucleic acid strand and a fifth nucleic acid strand are contacted to the ligated oligonucleotide immobilized on the array in the second cycle (e.g., as shown in FIG. 4). In some aspects, a sequence (e.g., splint sequence) on the second nucleic acid strand hybridizes to a third hybridization region in the fifth nucleic acid strand (e.g., splint). The fourth nucleic acid strand comprises a fourth nucleic acid sequence that hybridizes to a fourth hybridization region in the fifth nucleic acid strand. The fourth nucleic acid strand comprises a barcode sequence or portion thereof, such as a spatial barcode sequence. The fourth nucleic acid strand comprises a second primer sequence and a second UMI. As described above, the fourth nucleic acid strand is ligated to the ligated oligonucleotide (e.g., second nucleic acid strand) immobilized on the substrate. In some embodiments, the fourth nucleic acid strand and/or fifth nucleic acid strand is contacted with the substrate simultaneously or sequentially with a single strand annealing protein. In some embodiments, the single strand annealing protein is bound to the fourth and/or fifth nucleic acid strand. In some embodiments, single strand annealing proteins (e.g., DrB or RecT) promote ligation and/or binding of the fourth nucleic acid strand to the ligated oligonucleotide (e.g., second nucleic acid strand) immobilized on the substrate. The ligated oligonucleotide product immobilized on the substrate thus comprises a first nucleic acid sequence, a second nucleic acid sequence, and a fourth nucleic acid sequence.

In some embodiments, the method provided herein further comprises performing one or more additional ligations to generate a spatial array. For example, the method further comprises providing a sixth nucleic acid strand and a seventh nucleic acid strand in a third cycle. The fourth nucleic acid comprises a splint sequence that hybridizes to a sixth hybridization region in the seventh nucleic acid strand. The sixth nucleic acid strand comprises a sixth nucleic acid sequence that hybridizes to a seventh hybridization region in the seventh nucleic acid strand. The fourth nucleic acid strand is ligated to the sixth nucleic acid strand, using methods described herein. In some embodiments, single strand annealing proteins (e.g., DrB or RecT) bound to the fourth and/or sixth nucleic acid strands promote ligation and/or binding of the fourth nucleic acid strand to the ligated oligonucleotide (e.g., second nucleic acid strand) immobilized on the substrate. The ligated oligonucleotide product immobilized on the substrate thus comprises a first nucleic acid sequence, a second nucleic acid sequence, a fourth nucleic acid sequence and a sixth nucleic acid strand. In some embodiments, the polynucleotides (e.g., first nucleic acid sequence, a second nucleic acid sequence, a fourth nucleic acid sequence and a sixth nucleic acid strand) comprise barcode sequences (e.g., spatial barcode sequences), and various domains such as, UMIs, functional domains (e.g., sequencing handle), and/or cleavage domains.

In some embodiments, provided herein is a method of generating a spatial array comprising performing N cycles of single strand annealing protein-assisted ligation to generate a spatial array. In some embodiments, each of the N cycles comprises providing a second nucleic acid strand hybridized to a splint (e.g., a partially double-stranded nucleic acid strand and splint complex) and ligating the nucleic acid strand to a first nucleic acid strand immobilized on the substrate using the splint as a template, and a single strand annealing protein. In some embodiments, each of the N cycles comprises ligating a second nucleic acid strand to a first nucleic acid strand or strands immobilized in a different region or set of regions on the substrate, wherein the second nucleic acid strands are the same or different for different regions. In some embodiments, N is an integer of 2 or greater. In some embodiments, N is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater than 50. In some embodiments, the splint and second nucleic acid strand are contacted with the substrate together with the single strand annealing protein. In some embodiments, the single strand annealing protein is contacted with the substrate before contacting the substrate with the second nucleic acid strand and splint. In some embodiments, the single strand annealing protein is contacted with the substrate after contacting the substrate with the second nucleic acid and splint. In some embodiments, the splint and/or second nucleic acid strand are bound to the single strand annealing protein before contacting the substrate.

Typically, for spatial array-based methods, a substrate functions as a support for direct or indirect attachment of capture probes to features of the array. A feature can be an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication Nos. 2020/0277663, 2022/0010367, 2023/0143569, and 2023/0159995. Exemplary features and geometric attributes of an array can be found in Sections (II)(d)(i), (II)(d)(iii), and (II)(d)(iv) of WO 2020/176788 and/or U.S. Patent Application Publication Nos. 2020/0277663, 2022/0010367, 2023/0143569, and 2023/0159995.

In some embodiments, the array comprises an arrangement of a plurality of features, e.g., each comprising one or more oligonucleotide molecules (e.g., first nucleic acid strands). In some embodiments, the array comprises different oligonucleotide molecules in different features. In some embodiments, oligonucleotide molecules on the substrate are immobilized in a plurality of features. In some embodiments, ligated oligonucleotides within the same feature are typically the same, whereas ligated oligonucleotides occupying different features are mostly different from one another. In some instances, the same first nucleic acid strands are immobilized in a first feature and a second feature, and different second nucleic acid strands are ligated to the first nucleic acid strands in the first and second feature, respectively, thereby generating different ligated oligonucleotides in the first feature and the second feature. In some embodiments, the nucleic acid sequences received by the first nucleic acid strands in feature(s) on the substrate in cycle I and in feature(s) in cycle J are different, wherein I and J are integers and 1≤I<J≤N. In some embodiments according to any one of the methods described herein, the nucleic acid sequences in a feature of the substrate receive a second barcode sequence in one of the cycles in round K, wherein K is an integer and 1≤K, and nucleic acid sequences in the feature comprising the second barcode sequence receive a fourth barcode sequence in one of the cycles in round (K+1), thereby forming ligated oligonucleotide molecules comprising the second and third barcode sequences.

In some aspects, a nucleic acid strand comprises a capture sequence complementary to a sequence on a target nucleic acid (e.g., a poly-T sequence complementary to a poly-A sequence of an mRNA). In some embodiments, the method comprises ligating a nucleic acid strand comprising a capture sequence to an immobilized nucleic acid strand on a substrate to generate an immobilized capture probe. A capture probe can be any molecule capable of capturing (directly or indirectly) a nucleic acid strand corresponding to an analyte (e.g., target nucleic acid) in a biological sample. In some embodiments, a sequence of a nucleic acid strand (e.g., fourth, fifth, or sixth nucleic acid strand) comprises a capture sequence complementary to a sequence on a target nucleic acid. In some embodiments, the capture probe includes a barcode or barcodes (e.g., a spatial barcode and/or a UMI and a capture domain). In some embodiments, the barcode is generated by multiple rounds of single strand annealing protein-assisted ligation. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication Nos. 2020/0277663, 2022/0010367, 2023/0143569, and 2023/0159995.

In some embodiments, the spatially-barcoded capture probes described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence). The spatially-barcoded arrays generated according to the methods described herein can be applied for capture and analysis of endogenous nucleic acids, probes, or products thereof that are migrated to the array from a biological sample. In some examples, a nucleic acid ligation product (or a derivative thereof) can be captured by the a nucleic acid molecules (e.g., first nucleic acid strand) comprising the capture sequence (e.g. capture probes) and associated with a spatial barcode, optionally amplified, and sequenced, thus determining the location of the target nucleic acid in the biological sample. In some cases, the spatially-barcoded capture probes can be attached to functional sequences described herein (such as sequence specific flow cell attachment sequences) prior to analysis. In some cases, the spatially barcoded analyte (or a product or derivative thereof) can be released from the array prior to analysis. In some embodiments, the methods disclosed herein comprise releasing the spatially barcoded ligated oligonucleotide from the substrate. In some cases, the spatially barcoded analyte can be further processed and subjected to one or more reactions prior to analysis (e.g., extension, amplification, or other reactions).

In some embodiments, the method comprises sequencing all or a portion of the ligated probe or an amplification or extension product thereof. In some embodiments, the amplification or extension products of the ligated oligonucleotides comprise spatial barcode sequences or complements thereof that identify locations of the captured ligated oligonucleotides on the array. In some embodiments, the amplification or extension product of the ligated probe comprises a spatial barcode sequence or complement thereof that identifies a location of the captured ligated probe on the array.

### C. Generation of Barcoded Molecules

In some aspects, the present disclosure provides a method for generating a barcoded molecule by single strand annealing protein-assisted ligation. In some embodiments, the barcoded molecule is a cell or bead such as a gel bead. In some embodiments, the present disclosure provides a method for generating barcoded molecules within a cell (e.g., barcoded antibodies or cDNA). In some instances, the method comprises performing multiple rounds of ligation to generate a barcoded molecule. At least one, two, three, four or more rounds of ligation can be performed in the presence of a single strand annealing protein. In some embodiments, the single strand annealing protein is DdrB or an engineered derivative or variant thereof. In some embodiments, the single strand annealing protein is RecT or an engineered derivative or variant thereof. In some embodiments, the single strand annealing protein is Lambda phage Redβ of an engineered derivative or variant thereof.

In some embodiments, the method comprises combinatorially assembling a plurality of barcoded molecules (e.g., barcoded cells, beads, proteins, or nucleic acids) by coupling one or more first nucleic acid strands to each of the plurality of molecules and performing multiple rounds of ligation of additional nucleic acid strands to the first nucleic acid strands. In some embodiments, one or more rounds of the ligation in the combinatorial ligation scheme are performed in the presence of a single strand annealing protein. In some embodiments, two, three, four, or more rounds of ligation are performed in the presence of a single strand annealing protein. In combinatorial ligation schemes, the efficiency of each round of ligation contributes to the overall efficiency of generating full-length oligonucleotides. For example, if N rounds of ligation are performed to attach N nucleic acid barcode subunits to a first nucleic acid strand, the efficiency of generating full-length oligonucleotides comprising all N nucleic acid barcode subunits is equal to the efficiency of each round raised to the power N, or E^{N} where E=efficiency of each round of ligation. In a particular example, if the efficiency of ligation for each round is 80%, the efficiency of generating full length oligonucleotides in three rounds of ligation is 0.8³, or about 51%. Thus, it is highly desirable to increase the ligation efficiency for methods comprising multiple rounds of ligation, such as in combinatorial barcoding schemes. In some aspects, the present disclosure addresses this need by providing a method for increased ligation efficiency using single strand annealing proteins.

Multiple ligation reactions used in the preparation of ligated oligonucleotide molecules according to the methods provided herein may be performed in a combinatorial fashion. Combinatorial approaches permit the generation of a high diversity of barcoded molecules (e.g., barcoded beads) using a reduced number of different nucleic acid molecules in each individual ligation step, by performing multiple ligations in a combinatorial fashion. A combinatorial scheme involves assembling multiple molecular segments or sequences (e.g., nucleic acid barcodes or portions thereof) to provide a larger molecule (e.g., a ligated molecule comprising two or more molecular segments or sequences). In some embodiments, a first nucleic acid strand is immobilized on a substrate, and the first nucleic acid strand is ligated to a second nucleic acid strand using a third nucleic acid strand as a template to generate a ligated oligonucleotide, wherein a single strand annealing protein is bound to the first, second, and/or third nucleic acid strand. In some embodiments, a fourth nucleic acid strand is ligated to the ligated oligonucleotide using a fifth nucleic acid strand as a template to generate a further extended oligonucleotide immobilized on the bead (e.g., as illustrated in FIG. 4). In some embodiments, 2, 3, 4, 5, 10, or more ligations are performed to generate a full length oligonucleotide.

In some embodiments, the method comprises performing multiple rounds of ligation in a split-pool combinatorial ligation scheme. In some instances, the method comprises providing a plurality of partitions, wherein individual partitions within the plurality of partitions each comprise a plurality of beads comprising immobilized first nucleic acid strands. In some embodiments, the method comprises providing each of at least a portion of the partitions with a different second nucleic acid strand and a splint (third nucleic acid strand), and a single strand annealing protein. In some embodiments, the first nucleic acid strands individually comprise a common sequence such as a primer sequence (e.g., an R1 primer sequence). In some embodiments, the first nucleic acid strands each comprise a unique molecular identifier (UMI). In some embodiments, the first nucleic acid strands comprise a first nucleic acid sequence that hybridizes to the splint, wherein the first nucleic acid sequence is common among the first nucleic acid strands. In some embodiments, the splint hybridizes to a common sequence in the second nucleic acid strands (e.g., a universal splint having the same nucleic acid sequence can be used to ligate a plurality of different second nucleic acid strands to a plurality of first nucleic acid strands).

In some embodiments, multiple partitions are pooled together to form a pooled mixture, wherein the mixture comprises beads functionalized with a plurality of different second nucleic acid strands. The mixture can then be split into a plurality of partitions, wherein individual partitions within the plurality of partitions each comprise a plurality of beads functionalized with different second nucleic acid strands (e.g., by random chance). A second round of ligation is performed by providing each of at least a portion of the partitions with a different fifth nucleic acid strand and a splint (fourth nucleic acid strand), and a single strand annealing protein. After the ligation, the partitions can be pooled and split again to randomize the barcoded beads contained within each partition, providing a method for combinatorial barcoding.

In some embodiments, the method comprises providing a partition A comprising a plurality of beads comprising a first nucleic acid strand immobilized to the beads, a second oligonucleotide A, a splint that hybridizes to the first nucleic acid strand and the second oligonucleotide A, and a single strand annealing protein; and a partition B comprising a plurality of beads comprising a first nucleic acid strand immobilized to the beads, a second oligonucleotide B, a splint that hybridizes to the first nucleic acid strand and the second oligonucleotide B, and a single strand annealing protein. Second oligonucleotides A are ligated to the first nucleic acid strands of partition A, and second oligonucleotides B are ligated to the first nucleic acid strands of partition B to generate differently barcoded beads in partitions A and B. Subsequent to this first ligation step, beads of partition A and beads of partition B are pooled together. The pooled mixture comprising beads functionalized with second oligonucleotide A and beads functionalized with second oligonucleotide B. The pooled mixture is then partitioned between partitions C and D. Partitions C and D therefore each include beads functionalized with second oligonucleotide A and beads functionalized second oligonucleotide B. Partition C includes third oligonucleotides C and partition D includes third oligonucleotides D. Third oligonucleotides C are ligated to the beads of partition C to generate beads functionalized with A and C and beads functionalized with B and C, and third oligonucleotides D are ligated to the beads of partition D to generate beads functionalized with A and beads functionalized with B and D, optionally wherein each hybridization and/or ligation is performed in the presence of a single strand annealing protein. Subsequent to this second ligation step, in beads of partition C and beads of partition D can be pooled together. The resulting pooled mixture comprises beads functionalized with A and C, B and C, A and D, and B and D. The differentially functionalized beads may be divided amongst different partitions subjected to further ligation rounds, optionally in the presence of a single strand annealing protein. After generating the desired barcode diversity, the differentially functionalized beads may be partitioned and used for analysis of different analytes. For example, a single bead of the collection of beads may be co-partitioned with a single biological particle (e.g., a cell) comprising an analyte and one or more reagents. A plurality of partitions may be similarly generated (e.g., as described herein) such that each partition includes a bead having attached thereto a differently functionalized barcode molecule.

In some embodiments, the method comprises performing at least two, three, four, or more rounds of ligation, wherein each round of ligation is performed using at least 10, 20, 30, 40, 60, 70, 80, 90, 100, or more different nucleic acid strands (e.g., comprising different nucleic acid barcodes or portions thereof) in a corresponding number of partitions, followed by pooling the partitions and splitting the pooled mixture into a plurality of partitions for the subsequent round of ligation.

In some cases, a combinatorial ligation method may comprise immobilizing a plurality of beads to a plurality of supports (e.g., wells of a well plate). The first ligation step may involve providing a plurality of second nucleic acid strands to each partition and promoting a ligation reaction using a splint and a single strand annealing protein. Rather than pooling the plurality of beads from each partition, excess material may be washed out of each partition and a plurality of subsequent nucleic add molecules (e.g., fourth nucleic acid strands and splints (fifth nucleic acid strands) in FIG. 4) and a single strand binding protein washed into each partition for a second ligation reaction. Accordingly, the methods of the present disclosure may comprise moving beads between partitions or moving fluids between partitions.

A bead may be porous, non-porous, solid, semi-solid, semi-fluidic, fluidic, and/or any combination thereof. In some instances, a bead may be dissolvable, disruptable, and/or degradable. In some cases, a bead may not be degradable. In some embodiments, the bead is a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible. A bead may be of any suitable shape. Examples of bead shapes include, but are not limited to, spherical, non-spherical, oval, oblong, amorphous, circular, cylindrical, and variations thereof.

In some examples, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., first and/or second nucleic acid strand), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead. In some embodiments, the nucleic acid molecule sequence (e.g., first and/or second sequence) comprises a partition barcode sequence, a capture sequence, and optionally additional unique molecular identifier (UMI). In some embodiments, the first and/or second sequences may have sequences complementary to short oligonucleotides (e.g., nucleic acid splint) to enable additional ligation steps. In some aspects, the first and/or second sequences may have sequences that enable building a combinatorial barcode (e.g., ligating additional polynucleotides comprising barcodes to the first and/second sequences). In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise a R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

In some embodiments, the polynucleotides immobilized on the bead (e.g., first nucleic acid strands or ligated oligonucleotides generated on the bead) may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer binding sequence (such as an R1, R2, or partial R1 or R2 sequence).

### III. Compositions, Kits, And Systems

In some embodiments, disclosed herein is a composition that comprises a complex containing a target nucleic acid, a probe, e.g., any one of the probes described herein (for example, a circularizable probe), and a single strand annealing protein bound to the probe. In some embodiments, disclosed herein is a composition that comprises a complex containing a target nucleic acid, one or more nucleic acid strands, e.g., any one of nucleic acid strands described herein (for example, a first and/or second nucleic acid strands), and a single strand annealing protein bound to the one or more nucleic acid strands.

In some aspects, provided herein are protein bound-polynucleotides for improving nucleic acid ligation of the polynucleotides (e.g., probes and probe sets, nucleic acid molecules such as nucleic acid strands) to target nucleic acids, such as messenger RNA (mRNA) sequences. The protein-bound polynucleotides permit improved ligation efficiencies and precision during in situ detection of targets in highly complex samples, for example, in intact biological tissue containing numerous different mRNA sequences, improving-image based quantification of nucleotide expression at subcellular resolution. The protein-bound polynucleotides disclosed herein provide advantages of increased ligation efficiency to target nucleic acids, for in situ amplification and detection to occur in highly multiplexes in situ detection and single cell profiling assays. The exemplary polynucleotides provide advantages of improved ligation and fidelity in generating spatial arrays for analyzing spatial information of target nucleic acids.

In some embodiments, disclosed herein is a composition that comprises an amplification product containing monomeric units of a sequence complementary to a sequence of a probe (e.g., a circularizable probe). In some embodiments, the amplification product is formed using any one of the target nucleic acids, probes (e.g., circularizable probes) and any one of the amplification techniques described herein.

In some embodiments, disclosed herein is a composition that comprises a complex containing a nucleic acid strand immobilized to a substrate (e.g., array slide), one or more nucleic strands bound to a target nucleic acid, and a single strand annealing protein bound to one or more of the nucleic acid strands.

Also provided herein are kits, for example comprising one or more ligatable nucleic acid strands, a single strand annealing protein, and reagents for performing the methods provided herein, for example reagents required for one or more steps comprising hybridization, cleavage, ligation, amplification, detection, sequencing, and/or sample preparation as described herein. In some embodiments, the kit further comprises a target nucleic acid. In some embodiments, any or all of the polynucleotides are DNA molecules. In some embodiments, the target nucleic acid is a messenger RNA molecule. In some embodiments, the kit further comprises a single strand annealing protein, for instance binding to the probes or nucleic acid molecules. In some embodiments, the single strand annealing protein is DdrB. In some embodiments, the single strand annealing protein is RecT. In some embodiments, the kit further comprises a ligase, for instance for forming a ligated circular probe from the circularizable probe or for forming a ligated linear oligonucleotide from nucleic acid strands hybridized to the target nucleic acid. In some embodiments, the ligase has DNA-splinted DNA ligase activity. In some embodiments, the kit further comprises a polymerase, for instance for performing amplification of the circularizable probe. In some embodiments, the polymerase is capable of using the ligated circular probe as a template for amplification. In some embodiments, the kit further comprises a primer for amplification.

Disclosed herein in some aspects is a kit comprising a first nucleic acid strand, a second nucleic acid strand, and a third nucleic acid strand, wherein the first nucleic acid strand comprises a first sequence that hybridizes to a first hybridization region of the third nucleic acid strand, and the second nucleic acid strand comprises a second sequence that hybridizes to a second hybridization region of the third nucleic acid strand, wherein the first nucleic acid strand, second nucleic acid strand and/or the third nucleic acid strand are/is bound to a single strand annealing protein.

Disclosed herein in some aspects is a kit comprising probe or a probe set comprising a first sequence, wherein the probe hybridizes to a first hybridization region of target nucleic acid and a second sequence hybridized to a second hybridization region of the target nucleic acid, wherein the first sequence and/or the second sequence is/are bound to a single strand annealing protein.

In some aspects, disclosed herein is a kit comprising a partition comprising (i) a single biological particle, wherein the single biological particle comprises target nucleic acid and the second nucleic acid strand hybridized to the target nucleic acid, and (ii) a bead comprising the first nucleic acid strand comprising a partition barcode sequence and capture sequence, wherein the first nucleic acid strand is bound to a single strand annealing protein.

In some aspects, disclosed herein is a kit comprising a second nucleic acid strand, and a third nucleic acid strand, wherein the second nucleic acid strand comprises a second sequence that hybridizes to a second hybridization region of the third nucleic acid strand, and a first nucleic acid strand comprises a first sequence comprising: (i) a capture domain capable of capturing the third nucleic acid strand, and (ii) a spatial barcode sequence corresponding to the position of the first nucleic acid strand on the first substrate, wherein the first nucleic acid strand is immobilized on a substrate and wherein the first nucleic acid strand, second nucleic acid strand and/or the third nucleic acid strand are/is bound to a single strand annealing protein.

In some aspects, disclosed herein is a kit for use in a method for nucleic acid ligation, comprising: a) providing a first nucleic acid strand, a second nucleic acid strand, and a third nucleic acid strand; wherein the first nucleic acid strand comprises a first sequence that hybridizes to a first hybridization region of the third nucleic acid strand, and the second nucleic acid strand comprises a second sequence that hybridizes to a second hybridization region of the third nucleic acid strand, wherein the first nucleic acid strand, second nucleic acid strand and/or the third nucleic acid strand are/is bound to a single strand annealing protein; and b) ligating the first nucleic acid strand and second nucleic acid strand using the third nucleic acid strand as template to generate a ligated oligonucleotide comprising the first sequence and the second sequence.

In some aspects, disclosed herein is a kit for use in a method for nucleic acid ligation, comprising: a) contacting a biological sample with a probe or a probe set comprising a first sequence, wherein the probe hybridizes to a first hybridization region of target nucleic acid; b) ligating the first sequence of the probe or probe set to a second sequence hybridized to a second hybridization region of the target nucleic acid in the presence of a single strand annealing protein bound to the first sequence and/or the second sequence, wherein the second hybridization region is adjacent to the first hybridization region in the target nucleic acid, wherein the second sequence is a part of the probe or probe; and c) detecting the ligated probe or product thereof comprising the first sequence and second sequence or a product thereof.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any one of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### IV. Applications

In some aspects, the provided embodiments can be applied in an in situ method of analyzing nucleic acid sequences, such as an in situ transcriptomic analysis, for example from intact tissues or samples in which the spatial information has been preserved. In some aspects, the embodiments can be applied in a spatial array. In some aspects, the embodiments can be applied in a single-cell profiling assay. In some aspects, the embodiments can be applied in an imaging or detection method for multiplexed nucleic acid analysis. In some aspects, the provided embodiments can be used to improve ligation and/or binding of probes to target nucleic acids.

In some aspects, the provided embodiments can be applied in a method of processing nucleic acid strands to generate ligated oligonucleotides, such as in the generation of nucleic acid arrays and/or barcoded nucleic acid molecules (e.g., barcoded nucleic acid molecules immobilized on a substrate). In some aspects, the provided embodiments can be applied in a method of generating barcoded nucleic acid molecules by combinatorial ligation (e.g., combinatorial ligation of nucleic acid barcode subunits to nucleic acid strands immobilized on an array or combinatorial ligation according to a split-pool method).

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### V. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “oligonucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.”

A “hybridization complex” as used herein may comprise one, two, or more strands or separate molecules. A hybridization complex that comprises three or more strands or separate molecules does not necessarily comprise direct hybridization between every possible pairwise combination thereof, so long as at least two molecules or strands are directly hybridized to each other, or are in the process of binding to or unbinding from each other, at a given time.

“Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tm for the specific sequence at a defined ionic strength and pH. The melting temperature Tm can be the temperature at which a population of double-stranded nucleic acid molecules becomes half dissociated into single strands. Several equations for calculating the Tm of nucleic acids can be used. As indicated by standard references, a simple estimate of the Tm value may be calculated by the equation, Tm=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tm.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any one of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

In some aspects, conservative amino acid substitutions can frequently be made in a protein without altering either the conformation or the function of the protein. Proteins of the invention can comprise 1, 2, 3, 4, 5, 6, 7, 8, 9, 10, 11, 12, 13, 14, 15 conservative substitutions. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa.

Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g., U.S. Pat. No. 8,562,989; pages 13-15 “Biochemistry” 2^{nd} ED. Lubert Stryer ed (Stanford University); Henikoff et al., PNAS 1992 Vol 89 10915-10919; Lei et al., J Biol Chem 1995 May 19; 270(20):11882-6).

An amino acid substitution may include replacement of one amino acid in a polypeptide with another amino acid. Amino acid substitutions may be introduced to generate a modified AP50 polymerase as described herein.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some contexts, conservative substitutions can involve the exchange of a member of one of these classes for another member of the same class. In some contexts, non-conservative amino acid substitutions can involve exchanging a member of one of these classes for another class. In some contexts, particular substitutions can be considered “conservative” or “non-conservative” depending on the stringency and context and environment of the particular residue in primary, secondary and/or tertiary structure of the protein.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “adjacent” as used herein includes but is not limited to being directly linked by a phosphodiester bond. For example, “adjacent” nucleotides or regions on a nucleic acid such as a probe may be separated by a number of nucleotides. For instance, a toehold region and an interrogatory region adjacent to each other may be separated by 0, 1, 2, 3, 4, 5, 6, 7, 8, 9, 10 or more nucleotides in a probe.

As used herein, the term “antibody” refers to an antibody molecule of any class, or any sub-fragment thereof, such as a Fab.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

The term “each,” when used in reference to a collection of items, is intended to identify an individual item in the collection but does not necessarily refer to every item in the collection, unless expressly stated otherwise, or unless the context of the usage clearly indicates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following example is included for illustrative purposes only and is not intended to limit the scope of the present disclosure.

### Example 1: Improved Ligation Efficiency in the Presence of Single Strand Annealing Protein DdrB

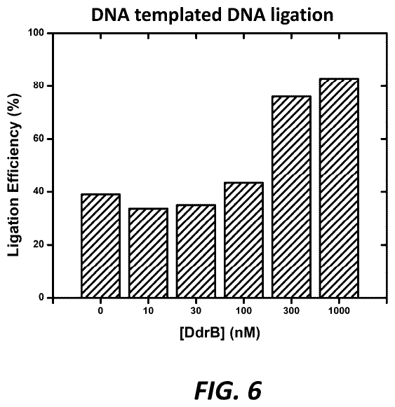

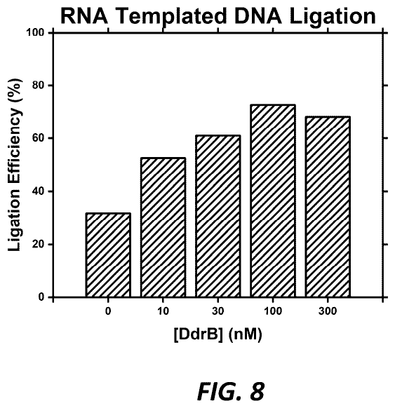

This example describes results from ligation assays of DNA polynucleotides using DNA templates (e.g., DNA templated DNA ligation, FIGS. 5A-5B and 6) and RNA templates (e.g., RNA templated DNA ligation, FIGS. 7A-7B and 8) with or without a single strand annealing protein.

As shown in FIG. 5A, a 5′-fluorescein amidite (FAM) labeled DNA probe (first nucleic acid strand) was incubated with the single strand annealing protein DdrB. A second 5′ phosphorylated DNA probe (second nucleic acid strand) was annealed to the complementary DNA template (third nucleic acid strand). The DdrB bound FAM-labeled DNA probe was additionally incubated with the annealed complex of second nucleic acid strand and third nucleic acid strand. The ligation reactions were performed with 50 nM final concentration of 5′-FAM labeled DNA probe, 60 nM final concentrations of 5′-phosphorylated unmodified DNA and DNA template. Reactions were incubated in ligation buffer containing 1×T4 RNA ligase2 buffer, 3 nM ligase enzyme and DdrB protein at varying concentrations of 0, 10 nM, 30 nM, 100 nM, 300 nM and 1000 nM at 37 C. for 30 minutes in a total volume of 20 μL. The resulting sample comprised a mixture of unreacted starting material, including the FAM labeled probe (as shown with an arrow in FIG. and ligated product (as shown with double arrows in FIG. 5B).

The products were denatured, separated by capillary electrophoresis and detected by fluorescence (FIG. 5B). As shown in FIG. 5B, the combination of ligase and DdrB promoted the ligation. FIG. 6 depicts the ligation efficiency as measured by the percentage of the ligated fraction of DNA using different concentrations of DdrB protein. As shown in FIG. 6, as DdrB concentration was increased, the ligation efficiency of the DNA templated DNA ligation reaction improved significantly in the presence of DdrB at the tested concentrations of 100 nM, 300 nM, and 1000 nM in comparison to reactions without DdrB (0 nM), thus more of the probe was ligated and converted to the desired product.

For RNA templated DNA ligation reactions, a 5′-FAM labeled DNA probe (first nucleic acid strand) was incubated with single strand annealing proteins DdrB (FIG. 7A). A second phosphorylated DNA probe (second nucleic acid strand) was annealed to the complementary RNA template (third nucleic acid strand). The DdrB bound FAM-labeled DNA probe was additionally incubated with the annealed complex of the second nucleic acid strand and third nucleic acid strand. The ligation reactions were performed with 50 nM final concentration of 5′-FAM labeled DNA probe, 60 nM final concentration of 5′-phosphorylated unmodified DNA and DNA template. Reactions were incubated in ligation buffer containing 1×T4 RNA ligase2 buffer, 30 nM ligase enzyme and DdrB protein at varying concentrations of 0, 10 nM, 30 nM, 100 nM, 300 nM and 1000 nM at 37 C for 30 minutes in a total volume of 20 μL. The resulting sample comprises a mixture of unreacted starting material, including the FAM labeled probe (as shown with an arrow in FIG. 7B) and ligated product (as shown with double arrows in FIG. 7B). The products are denatured, separated by capillary electrophoresis and detected by fluorescence (FIG. 7B). As shown in FIG. 7B, the combination of ligase and DdrB promoted the ligation. FIG. 8 depicts the ligation efficiency as measured by the percentage of the ligated fraction of DNA using different concentrations of DdrB protein. As shown in FIG. 8, the ligation efficiency of the DNA templated RNA ligation reaction improved significantly in the presence of DdrB at the tested concentrations of 10 nM, 30 nM, 100 mM and 300 nM in comparison to reactions without DdrB (0 nM), thus more of the probe was ligated and converted to the desired product.

In conclusion, the use of single strand annealing proteins such as DdrB during annealing reactions significantly improves ligation efficiency as compared to reactions without DdrB.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for nucleic acid ligation, comprising:
a) providing a first nucleic acid sequence and a second nucleic acid sequence, wherein the first nucleic acid sequence hybridizes to a first hybridization region of a third nucleic acid sequence and the second nucleic acid sequence hybridizes to a second hybridization region of the third nucleic acid sequence,
wherein the first nucleic acid sequence, the second nucleic acid sequence, and/or the third nucleic acid sequence are/is bound to a single strand annealing protein; and
b) ligating the first nucleic acid sequence and the second nucleic acid sequence using the third nucleic acid sequence as a template to generate a ligated oligonucleotide comprising the first nucleic acid sequence and the second nucleic acid sequence.

2. The method of claim 1, wherein the first nucleic acid sequence and the second nucleic acid sequence are in the same nucleic acid molecule.

3. The method of claim 1 or claim 2, wherein the first nucleic acid sequence and the second nucleic acid sequence are a first end and a second end, respectively, of a circularizable probe.

4. The method of claim 1, wherein the first nucleic acid sequence and the second nucleic acid sequence are in different nucleic acid molecules.

5. The method of any of claims 1-4, wherein the single strand annealing protein promotes ligation of the first nucleic acid sequence and the second nucleic acid sequence.

6. The method of any of claims 1-5, wherein the single strand annealing protein promotes hybridization of the first nucleic acid sequence and/or the second nucleic acid sequence to the third nucleic acid sequence.

7. The method of any of claims 1-6, wherein ligation efficiency is increased by at least 10%, at least 20%, at least 30%, at least 40%, at least 50%, at least 60%, at least 70%, at least 80%, at least 90%, or at least 100% with the bound single strand annealing protein compared to ligation in the absence of the single strand annealing protein.

8. The method of any of claims 1-7, wherein the single strand annealing protein is DdrB or RecT.

9. The method of claim 8, wherein the concentration of DdrB protein used is at least 10 nM, at least 100 nM, at least 300 nM, at least 500 nM, at least 700 nM, or at least 1000 nM.

10. The method of any of claims 1-9, wherein the ligating is performed using a ligase, optionally wherein the ligase is a DNA ligase or an RNA ligase.

11. The method of any of claims 1-10, wherein the 3′ end of the first nucleic acid sequence is ligated to the 5′ end of the second nucleic acid sequence.

12. The method of any of claims 1-10, wherein the 5′ end of the first nucleic acid sequence is ligated to the 3′ end of the second nucleic acid sequence.

13. The method of any of claims 1-12, wherein the first nucleic acid sequence is in a probe, and the method comprises contacting a biological sample with the probe,
wherein the third nucleic acid sequence is in a target nucleic acid in the biological sample,
wherein the second nucleic acid sequence is in the same probe or is in a different probe that hybridizes to the target nucleic acid, wherein the ligating is performed in the biological sample, and
wherein the ligated oligonucleotide is a ligated probe comprising the first nucleic acid sequence and the second nucleic acid sequence.

14. The method of 13, wherein the method comprises detecting the ligated probe or a product thereof.

15. The method of claim 13 or 14, wherein the method comprises detecting the ligated probe or a product thereof at a location in the biological sample.

16. The method of any of claims 1-15, wherein the first hybridization region and the second hybridization region in the third nucleic acid sequence are directly linked by a phosphodiester bond.

17. The method of any of claims 1-15, wherein the first hybridization region and the second hybridization region in the third nucleic acid sequence are linked by one, two, three, four, five or more nucleic acid residues, and the method comprises performing a gap filling reaction with a polymerase using the third nucleic acid sequence as a template and/or hybridizing an oligonucleotide to the third nucleic acid sequence at a region between the first and second hybridization regions.

18. The method of any of claims 1-17, wherein the ligated oligonucleotide is a linear probe or a circular probe.

19. The method of any of claims 1-18, wherein the third nucleic acid sequence is RNA and the first nucleic acid sequence and/or second nucleic acid sequence comprises DNA.

20. The method of any of claims 1-19, wherein the first nucleic acid sequence and/or the second nucleic acid sequence comprises a ribonucleotide, optionally wherein the ribonucleotide is a 3′ end ribonucleotide of the first nucleic acid sequence or the second nucleic acid sequence that is ligated to the second nucleic acid sequence or the first nucleic acid sequence, respectively.

21. The method of any of claims 1-20, wherein the first nucleic acid sequence and/or the second nucleic acid sequence comprises no more than four consecutive ribonucleotides.

22. The method of any of claims 1-21, wherein the first nucleic acid sequence is in a first nucleic acid strand comprising a first barcode sequence, and/or wherein the second nucleic acid sequence is in a second nucleic acid strand comprising a second barcode sequence, wherein the first and second barcode sequences are of the same sequence or of different sequences.

23. The method of claim 22, wherein the first barcode sequence is in a 3′ overhang region of the first nucleic acid strand upon hybridization to the third nucleic acid sequence, and the second barcode sequence is in a 5′ overhang region of the second nucleic acid strand upon hybridization to the third nucleic acid sequence, or
wherein the first barcode sequence is in a 5′ overhang region of the first nucleic acid strand upon hybridization to the third nucleic acid sequence, and the second barcode sequence is in a 3′ overhang region of the second nucleic acid strand upon hybridization to the third nucleic acid sequence.

24. The method of any of claims 1-23, wherein the first nucleic acid sequence and/or the second nucleic acid sequence comprises a barcode sequence or a portion thereof.

25. The method of any of claims 13-24, wherein the biological sample is further contacted with a secondary probe that hybridizes to the ligated probe or a product thereof.

26. The method of claim 25, wherein the secondary probe is a detectably labeled probe comprising a detectable moiety.

27. The method of claim 25, wherein the secondary probe comprises an overhang region that does not hybridize to the ligated probe or product thereof.

28. The method of claim 27, wherein the method comprises contacting the biological sample with a detectably labeled oligonucleotide, wherein the detectably labeled oligonucleotide comprises a detectable moiety and a sequence that hybridizes to the overhang region of the secondary probe.

29. The method of claim 26 or claim 28, wherein the detectable moiety is a fluorophore.

30. The method of any of claims 25-29, wherein the secondary probe is bound to a single strand annealing protein, and wherein the single strand annealing protein promotes hybridization of the secondary probe to the ligated probe or product thereof.

31. The method of any of claims 28-30, wherein the detectably labeled oligonucleotide is bound to a single strand annealing protein, and wherein the single strand annealing protein promotes hybridization of the detectably labeled oligonucleotide to the secondary probe.

32. The method of any of claims 13-31, wherein the method comprises detecting the ligated probe or product thereof, wherein the detecting comprises determining a sequence of the probe, or a complementary sequence or a product of the probe.

33. The method of any of claims 13-32, wherein the target nucleic acid is at a location in a biological sample and the ligated probe is generated and optionally amplified at the location in the biological sample, and wherein the ligated probe and/or the product thereof is detected at the location in the biological sample, optionally wherein the product is a rolling circle amplification (RCA) product.

34. The method of any of claims 1-18 and 21-33, wherein the first nucleic acid sequence, the second nucleic acid sequence, and the third nucleic acid sequence are DNA.

35. The method of any of claims 1, 4-12, 16-24, and 34, wherein the second nucleic acid sequence and the third nucleic acid sequence are provided in a) as a partially double stranded duplex.

36. The method of any of claims 4-12, 16-24, and 34-35, wherein the first nucleic acid strand is immobilized on a substrate.

37. The method of claim 36, wherein the first nucleic acid strand comprises a primer sequence.

38. The method of claim 36 or claim 37, wherein the first nucleic acid strand comprises a unique molecular identifier (UMI).

39. The method of any of claims 36-37, wherein the first nucleic acid strand further comprises a barcode sequence or portion thereof.

40. The method of any of claims 36-39, wherein the second nucleic acid sequence comprises a barcode sequence or portion thereof, optionally wherein the barcode sequence is a spatial barcode associated with a location on the substrate.

41. The method any of claims 36-40, wherein the second nucleic acid strand further comprises a splint sequence, optionally wherein the splint sequence is common among a plurality of second nucleic acid strands immobilized on the substrate or on a plurality of substrates.

42. The method of claim 41, wherein the method further comprises providing a fourth nucleic acid strand and a fifth nucleic acid strand, wherein the splint sequence of the second nucleic acid strand hybridizes to a third hybridization region in the fifth nucleic acid strand, and the fourth nucleic acid strand comprises a fourth nucleic acid sequence that hybridizes to a fourth hybridization region in the fifth nucleic acid strand, optionally wherein the second, fourth, and/or fifth nucleic acid strand are/is bound to a single strand annealing protein.

43. The method of claim 42, wherein the fourth nucleic acid strand comprises a barcode sequence or portion thereof, optionally wherein the barcode sequence is a spatial barcode associated with a location on the substrate.

44. The method of 42 or 43, wherein the fourth nucleic acid strand comprises a second primer sequence.

45. The method of any of claims 42-44, wherein the fourth nucleic acid strand comprises a second unique molecular identifier sequence.

46. The method of any of claims 42-45, wherein the second nucleic acid strand is ligated to the fourth nucleic acid strand.

47. The method of claim 46, wherein the single strand annealing protein promotes ligation of the second nucleic acid strand to the fourth nucleic acid strand.

48. The method of any of claims 42-45, wherein the single strand annealing protein promotes hybridization of the second and/or fourth nucleic acid strand to the fifth nucleic acid strand.

49. The method of any of claims 42-48, comprising generating a ligated oligonucleotide comprising the first nucleic acid sequence, the second nucleic acid sequence, and the fourth nucleic acid sequence.

50. The method of any of claims 46-49, wherein the method comprises providing a sixth nucleic acid strand and a seventh nucleic acid strand, wherein the fourth nucleic acid strand comprises a splint sequence that hybridizes to a sixth hybridization region in the seventh nucleic acid strand, and the sixth nucleic acid strand comprises a sixth nucleic acid sequence that hybridizes to a seventh hybridization region in the seventh nucleic acid strand, optionally wherein the fourth, sixth, and/or seventh nucleic acid strand are/is bound to a single strand annealing protein.

51. The method of claim 50, wherein the fourth nucleic acid strand is ligated to the sixth nucleic acid strand.

52. The method of any of claims 42-51, comprising N cycles of ligation of different nucleic acid strands to the first nucleic acid strand to generate an immobilized ligated nucleic acid, wherein N is an integer of 2 or greater, and one of the N cycles comprises the providing in step a) and ligating in step b).

53. The method of claim 52, wherein at least 2, 3, or 4 of the N cycles are performed in the presence of the single strand annealing protein.

54. The method of any of claims 42-53, wherein a plurality of first nucleic acid strands are immobilized in a plurality of features on the substrate.

55. The method of claim 54, wherein the nucleic acid sequences received by the first nucleic acid strands on the substrate in cycle I and in cycle J are different, wherein I and J are integers and 1≤I<J≤N.

56. The method of any of claims 52-55, wherein the immobilized ligated nucleic acid comprises a capture sequence.

57. The method of any of claims 36-56, wherein the substrate is a planar substrate.

58. The method of any of claims 36-53 and 56, wherein the substrate is a bead.

59. The method of claim 58, wherein the bead is a gel bead.

60. The method of claim 58 or 59, wherein the method comprises ligating a second nucleic acid strand A to the immobilized first nucleic acid strand for a plurality of beads in a partition A, and ligating a different second nucleic acid strand B to the immobilized first nucleic acid strand for a plurality of beads in a partition B;
pooling partitions A and B; and
splitting the pooled partitions into partitions C and D before performing one or more additional cycles of ligating nucleic acid strands to the immobilized nucleic acid strands in the partitions, wherein each of the cycles of ligating is performed in the presence of a single strand annealing protein.

61. A method for analyzing a target RNA, comprising:
a) contacting the target RNA with a circularizable probe or probe set comprising a first nucleic acid sequence that hybridizes to a first hybridization region of the target RNA;
b) ligating the first nucleic acid sequence of the circularizable probe or probe set to a second nucleic acid sequence hybridized to a second hybridization region of the target RNA in the presence of a single strand annealing protein bound to the first nucleic acid sequence and/or the second nucleic acid sequence, wherein the second nucleic acid sequence is a part of the circularizable probe or probe set, wherein the second hybridization region is adjacent to the first hybridization region in the target RNA, thereby generating a circularized probe comprising the first nucleic acid sequence and the second nucleic acid sequence; and
c) detecting the circularized probe or a product thereof, optionally wherein the product of the circularized probe is a rolling circle amplification product.

62. A method for analyzing a biological sample, comprising:
contacting a target nucleic acid in the biological sample with a probe, wherein the probe is bound to a single strand annealing protein and the probe comprises a recognition sequence that hybridizes to a target sequence in the target nucleic acid, and
detecting a signal associated with the probe at a location in the biological sample, thereby detecting the target nucleic acid at the location in the biological sample.

63. The method of claim 62, wherein the probe is a detectably labeled probe.

64. The method of claim 63, wherein the probe comprises a binding site for a detectably labeled oligonucleotide, or for an intermediate probe that binds directly or indirectly to a detectably labeled oligonucleotide, and the method comprises contacting the biological sample with the detectably labeled oligonucleotide and/or intermediate probe, thereby associating the signal with the probe hybridized to the target nucleic acid.

65. The method of any of claims 63-64, wherein the target nucleic acid is a DNA concatemer comprising multiple copies of the target sequence.

66. The method of any of claims 63-64, wherein the target nucleic acid is an RNA.

67. A method comprising generating a plurality of ligated oligonucleotides, wherein each ligated oligonucleotide is generated according to the method of any one of claims 1-35, and detecting the plurality of ligated oligonucleotides or products thereof.

68. The method of claim 67, wherein the plurality of ligated oligonucleotides or products thereof are detected at different locations in the biological sample.

69. The method of claim 67 or 68, wherein the detecting comprises:
contacting the biological sample with one or more detectably labeled probes that directly or indirectly bind to one or more barcode sequences or complements thereof in the plurality of ligated oligonucleotides or products thereof,
detecting signals associated with the one or more detectably labeled probes, and
removing the one or more detectably labeled probes.

70. The method of any of claims 67-69, wherein the detecting comprises:
contacting the biological sample with one or more intermediate probes that directly or indirectly bind to one or more barcode sequences or complements thereof in the plurality of ligated oligonucleotides or products thereof, wherein the one or more intermediate probes are detectable using one or more detectably labeled probes, and
detecting signals associated with the one or more detectably labeled probes.

71. The method of claim 70, further comprising removing the one or more intermediate probes and/or the one or more detectably labeled probes.

72. The method of claim 67, wherein the method comprises capturing the plurality of ligated oligonucleotides on an array.

73. The method of claim 72, wherein detecting the plurality of ligated oligonucleotides or products thereof comprises sequencing all or a portion of the captured ligated oligonucleotides or complements thereof, optionally wherein the captured ligated oligonucleotides or complements thereof are amplified.

74. The method of any of claims 13-32, wherein the method comprises capturing the ligated probe on an array.

75. The method of claim 74, comprising sequencing all or a portion of the captured ligated probe or a complement thereof, optionally wherein the captured ligated probe or complement thereof are amplified.

76. The method of claim 73, wherein the amplification products of the captured ligated oligonucleotides or complements thereof comprise spatial barcode sequences or complements thereof that identify locations of the captured ligated oligonucleotides on the array.

77. The method of claim 75, wherein the amplification product of the captured ligated probe or complement thereof comprises a spatial barcode sequence or a complement thereof that identifies the location of the captured ligated probe on the array.


SEQUENCE LISTING (5 sequences)

SEQ ID NO: 1 - protein, 269 aa, Escherichia coli
  REGION 1..269: note = RecT
MTKQPPIAKADLQKTQGNRAPAAVKNSDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFSFEFGLDEKLIHRPGENEDAPVTHVYAVARLKDGGTQFEVMTRKQIELVRSLSKAGNNGPWVTHWEEMAKKTAIRRLFKYLPVSIEIQRAVSMDEKEPLTIDPADSSVLTGEYSVIDNSEE

SEQ ID NO: 2 - protein, 260 aa, synthetic construct
  REGION 1..260: note = Lambda phage Red-Beta
STALATLAGKLAERVGMDSVDPQELITTLRQTAFKGDASDAQFIALLIVANQYGLNPWTKEIYAFPDKQNGIVPVVGVDGWSRIINENQQFDGMDFEQDNESCTCRIYRKDRNHPICVTEWMDECRREPFKTREGREITGPWQSHPKRMLRHKAMIQCARLAFGFAGIYDKDEAERIVENTAYTAERQPERDITPVNDETMQEINTLLIALDKTWDDDLLPLCSQIFRRDIRASSELTQAEAVKALGFLKQKAAEQKVAA

SEQ ID NO: 3 - protein, 188 aa, Deinococcus radiodurans
  REGION 1..188: note = DdrB (GenBank: QIP28318.1)
MLQIEFITDLGARVTVNVEHESRLLDVQRHYGRLGWTSGEIPSGGYQFPIENEADFDWSLIGARKWKSPEGEELVIHRGHAYRRRELEAVDSRKLKLPAAIKYSRGAKVSDPQHVREKADGDIEYVSLAIFRGGKRQERYAVPGGAAGNGQGRPAPQGQPAQARPQATAARPAARPPVQPGQEEETPF

SEQ ID NO: 4 - protein, 178 aa, Deinococcus geothermalis
  REGION 1..178: note = DdrB (Dgeo_0070)
MLHIEFITDLGAKVTVDVESADKLLDVQRQYGRLGWTSGEVPVGGYQFPLENEPDFDWSLIGARKWTNPEGEEMILHRGHAYRRRELEAVDSRKMKLPAAVKYSRGAKNTDPEHVREKADGEFEYVTLAIFRGGKRQERYAVPGSNRPQAGAPARSAATRAQGARPGAVAVQDEETPF

SEQ ID NO: 5 - protein, 178 aa, Deinococcus deserti
  REGION 1..178: note = DdrB (NCBI Reference Sequence: WP_012692241.1)
MLHIEFLTDLGAKVTVDVEHENQLLDVQRHYGRLGWTSGAVPTGGYQFPLDNEPDFDWSLIGARKWTSPDGEELVIHKGHAYRRRELEAVDSRKMKLPAAVKFSRGAKSTDPEHLREKADGEFEYVTLAIFRGGKRQERYAVPGGARPAAQSARPAPARAQAAARPAPVAVVDEETPF